(19) United States
(12) Patent Application Publication
(10) Pub. No.: US 2026/0200986 A1
(43) Pub. Date: Jul. 16, 2026

(54) THERAPEUTIC FUSION PROTEINS FOR TARGETING PATHOGENIC PROTEIN AGGREGATES FOR DEGRADATION

(71) Applicants: CAMBRIDGE ENTERPRISE LIMITED, Cambridge (GB); UNITED KINGDOM RESEARCH AND INNOVATION, Swindon (GB)

(72) Inventors: William Alexander MCEWAN, Cambridge (GB); Lauren Virginia Clare MILLER, Cambridge (GB); Leo C. JAMES, Cambridge (GB); Dean CLIFT, Cambridge (GB); Guido PAPA, Cambridge (GB)

(21) Appl. No.: 19/130,879

(22) PCT Filed: Nov. 15, 2023
(86) PCT No.: PCT/EP2023/081931

§ 371 Date: May 16, 2025

(30) Foreign Application Priority Data
Nov. 16, 2022 (GB) 2217084.9

(51) Int. Cl. C07K 14/00; A61K 38/00; C12N 9/10; C12N 15/86

(52) U.S. Cl. CPC C07K 14/001; C12N 9/104; C12N 15/86; A61K 38/00; C07K 2319/95; C12N 2710/10043; C12N 2740/15043; C12N 2750/14143; C12N 2830/008

(57) ABSTRACT

The invention relates to methods and materials for use in treating neurodegenerative diseases associated with pathological protein aggregates and provides fusion proteins comprising: (i) a first portion comprising a sequence of a protein which aggregates pathologically in neurodegenerative disease, such as tau protein, and (ii) a second portion comprising a RING-type E3 ubiquitin ligase, a component of a RING-type E3 ubiquitin ligase complex, or a domain of either. These fusion proteins have utility in selectively or preferentially targeting pathogenic protein aggregates for degradation.

### CROSS-REFERENCE TO RELATED APPLICATIONS

This patent application claims the benefit of priority of GB 2217084.9 filed on 16 Nov. 2022 and which is herein incorporated in its entirety.

### TECHNICAL FIELD

The present invention relates generally to methods and materials for use in treating neurodegenerative diseases associated with pathological protein aggregates by use of therapeutic polypeptides which inhibit formation of the aggregates, or increase degradation or clearance of the aggregates.

### BACKGROUND TO THE INVENTION

Neurodegenerative diseases (NDs) include highly debilitating illnesses, such as Alzheimer's (AD), Parkinson's disease (PD), amyotrophic lateral sclerosis, Huntington's disease, spinocerebellar ataxias, frontotemporal dementia (FTD), corticobasal degeneration, progressive supranuclear palsy, chronic traumatic encephalopathy, multiple system atrophy, dementia with Lewy bodies, and prion diseases (PrD).

Notwithstanding large differences in clinical manifestation and prevalence, NDs have many common features, including their chronic and progressive nature, increase of prevalence with age, destruction of neurons in specific areas of the brain, damage of the network of synaptic connections, and selective brain mass loss.

A common aetiology in these diseases is the progressive accumulation of misfolded protein aggregates in well-ordered structures, usually referred to as “amyloid”. Despite the fact that the protein aggregates involved in distinct NDs are different, the process of protein misfolding, its intermediates, end-products, and main features are remarkably similar (Soto, Claudio, and Sandra Pritzkow. “Protein misfolding, aggregation, and conformational strains in neurodegenerative diseases.” Nature neuroscience 21.10 (2018): 1332-1340).

Targeted inhibition or degradation of these aggregates has significant therapeutic application. The use of targeted protein degradation as a therapeutic strategy minimizes the off target effects of drugs and avoids or reduces systemic drug exposure (Wu, T, et al. (2020) Nature Structural & Molecular Biology, 27:605-614).

Cells have two main options for processing misfolded or malfunctioning proteins: the autophagy-lysosomal network and the ubiquitin-proteasome system (UPS).

Auto-lysosomes degrade their contents using acid hydrolases and the resulting cellular building blocks are released back into the cytosol to be recycled.

The proteasome targets polyubiquitinated proteins and breaks them down via proteolysis into short peptide fragments.

Several approaches have been explored to send intracellular target protein species for degradation. They include the intracellular expression of antibody fragments, proteolysis-targeting chimaeras (PROTACs), lysosome-targeting chimaeras (LYTACs) and hydrophobic tagging which is recognised by the UPS 6.

TRIM21 is a cytoplasmic antibody receptor and E3 ubiquitin ligase. Clustering of TRIM21 following binding to antibody-bound substrates causes TRIM21's N-terminal RING domain to become activated, promoting ubiquitin chain formation 7. Ubiquitin chain catalysis relies on the E2 enzymes Ube2w and Ube2n, which promote mono-ubiquitination of TRIM21 and K63-linked ubiquitin chain extension respectively. This activity results in the degradation of antibody-bound targets at the proteasome.

It has been reported that assemblies of tau can import anti-tau antibodies to the cytosol enabling TRIM21 to target tau aggregates for proteasomal degradation 8.

WO2012/010855 relates to compounds comprising: (a) a ligand which binds, directly or indirectly, specifically to an antigen of a pathogen, provided that said ligand is not the PRYSPRY domain of TRIM21; and (b) a RING domain and/or an inducer of TRIM21 expression. These are reported to have utility in treating pathogenic infection. Notwithstanding these earlier disclosures, it can be seen that providing novel materials and methods for treating neurodegenerative diseases associated protein aggregates by selectively targeting those aggregates would provide useful contributions to the art.

### DISCLOSURE OF THE INVENTION

The present inventors have devised a novel agent for selectively or preferentially targeting pathogenic protein aggregates for degradation which (purely for brevity) is termed ‘E3 ligase-bait’ herein.

E3 ligases of the RING type are characterized by the presence of a RING domain, which is the minimal element required to recruit E2~ubiquitin and stimulate ubiquitin transfer.

As explained in Balaji, Vishnu, and Thorsten Hoppe (F1000Research 9 (2020)) regulation of these ligases is provided in part by homotypic and heterotypic combination of E3 ligases into oligomeric (i.e. higher order assembly) ubiquitylation complexes. As explained in Errington, Wesley J., et al. “Adaptor protein self-assembly drives the control of a cullin-RING ubiquitin ligase.” Structure 20.7 (2012): 1141-1153, the RING superfamily consists of many hundreds of E3s that differ greatly in size, structure, subunit stoichiometry, and mode of regulation. Common within the RING superfamily, however, is a remarkable propensity to self-assemble into oligomers to enhance activity.

Some RING E3s are large multi-subunit complexes. Such E3 ligase complexes of the RING type typically comprise a number of components or subunits which can self assemble in vivo, which are typically a scaffold component, an adaptor component, a substrate recognition component, as well as a catalytic E3 ligase component.

Once assembled these RING-type E3 ubiquitin ligase complexes can facilitate the direct transfer of ubiquitin from E2-ubiquitin intermediates to the target protein.

The inventors have reasoned that proteins undergoing aggregation should promote E3 ligase assembly and thus enhance their degradation activity against those aggregating proteins.

In this system a so-called “bait” protein with a capacity to co-aggregate in vivo with target proteins is fused to an E3 ubiquitin ligase, or component of an E3 ubiquitin ligase complex, or domain of either. The formation of aggregates therefore incorporates both cellular pools of the target protein as well as the E3 ligase-bait construct.

When the fusion proteins are incorporated into the growing aggregate, the second portions are brought into proximity, where they may oligomerize and/or recruit other parts or co-factors of the complete E3 ubiquitin ligase complex if required. This leads to the ubiquitination and removal of assembled versions of the target in which the E3 ligase-bait is incorporated.

Purely by way of exemplification, the inventors fused full-length tau, bearing a pro-aggregation mutation, and the TRIM21 RING domain. The TRIM21 RING domain was selected as it is activated via clustering⁷.

Expression of tau-RING prevented tau seeded aggregation and removed existing tau aggregates via the ubiquitin-proteasome system.

By contrast, monomeric tau was not affected by tau-RING expression, consistent with tau-RING specifically associating with, and activating in the context of assembled forms of tau. Intravenous and stereotaxic injection of AAVs encoding tau-RING to a mouse model of tau pathology substantially reduced the number of cell bodies bearing tau aggregates and the total burden of insoluble tau aggregates in the brain. Additionally, delivery of the tau-RING construct resulted in behavioural improvements in the same mouse model, demonstrating the therapeutic benefit of removing the aggregates. Corresponding results were obtained with RING-baits based on Fused in sarcoma (FUS) protein bearing the P525L mutation.

The inventors further showed that within the fusion protein, components of E3 ligase complexes, which components may not themselves contain a RING domain, can nevertheless be effective in E3 ligase-baits, with the recruitment of subunits and clustering induced upon aggregation leading to degradation of bait-fusion containing aggregates. Purely by way of exemplification, they fused tau bait to VHL, a substrate-recognition component of cullin ligases, and observed the selective removal of tau aggregates from cells.

The findings described herein reveal that localisation by means of using the aggregating protein itself as a ‘bait’ is a novel strategy for the localisation and aggregation-induced assembly of E3 ubiquitin ligase activity that can be exploited for the removal of aggregated or assembled proteins.

Importantly, this strategy can be effective against both seeded aggregation and cell autonomous protein aggregation^{5 9}. Furthermore the invention can be applied to intraneuronal populations of aggregates, which may be particularly beneficial⁴.

In a first aspect there is provided a fusion protein comprising:
(i) a first portion comprising a sequence of a protein which aggregates pathologically in neurodegenerative disease, and
(ii) a second portion comprising an E3 ubiquitin ligase, or component of an E3 ubiquitin ligase complex, or domain of either.

A “fusion protein” or a “fusion polypeptide” (the terms are used interchangeably) refers to a polypeptide having two or more portions covalently linked together, where each of the portions is a polypeptide having a biological property.

In the present invention the first portion is derived from a protein which aggregates pathologically in neurodegenerative disease. For brevity herein this may be referred to as the “protein aggregation” portion or domain.

As explained above, the second portion comprises an E3 ubiquitin ligase, or component of an E3 ubiquitin ligase complex, or domain of either. Preferably the E3 ubiquitin ligase or complex is of the RING type. The second portion may or may not itself possess E3 ligase catalytic activity.

Both parts of the fusion protein are described in detail hereinafter.

The term “E3 ubiquitin ligase” and “E3 ligase” are used interchangeably herein.

Some E3 ligases become functional when part of larger complexes. These can comprise a number of components or subunits which can self assemble in vivo: these include: a substrate receptor (which may in turn comprise an adaptor complex and substrate recognition module such as VHL), a scaffold (e.g. a Cullin2 polypeptide) as well as a catalytic E3 ligase component which is a RING protein (i.e. RING or RING-finger domain containing protein) such as a TRIM or Rbx protein. The activated E3 ligase is capable of covalent transfer of ubiquitin to itself or to another protein.

Those skilled in the art are well aware of examples of scaffold components, adaptor components, substrate recognition components, and catalytic E3 ligase components (see e.g. Buetow, Lori, and Danny T. Huang. “Structural insights into the catalysis and regulation of E3 ubiquitin ligases.” Nature reviews Molecular cell biology 17.10 (2016): 626-642) By way of non-limiting example, scaffold components include: CUL1, CUL2, CUL3, CUL4A, CUL4B, CUL5, CUL7, CUL9; adaptor components include: SKP1, Elongin B, Elongin C, DDB1, BTB; substrate recognition components include FBXW1, FBXW11, FBXL1, FBXO1, VHL, LRR1, FEM1, KEAP1, SPOP, KLHL40, BTB6A, DCAF1, DCAF14, DDB2, CRBN; catalytic E3 ligase components include RBX1, RBX2.

A “domain” is a folded protein structure which retains its tertiary structure independently of the rest of the protein. Generally, domains are responsible for discrete functional properties of proteins, and in many cases may be added, removed or transferred to other proteins without loss of function of the remainder of the protein and/or of the domain. The RING domain of TRIM polypeptides is an example thereof.

Such fusions proteins may comprise, consist, or consist essentially of the portions described herein.

Such fusion proteins (polypeptides) may be provided in isolated, purified, or semi-purified form.

Because the fusion proteins contain heterologous sequences, which do not occur together in nature, they are non-naturally occurring.

The two portions of the fusion protein may be linked directly by a single peptide bond or through a peptide linker containing one or more amino acid residues. Generally, the two portions and the linker will be in reading frame with each other.

Typically, fusion proteins will be prepared by DNA recombination techniques standard in the art and may be referred to herein as recombinant fusion proteins.

The term “recombinant” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in manner different from its natural expression profile, and the like. Generally, recombinant nucleic acids, polypeptides, and cells based thereon, have been manipulated by man such that they are not identical to related nucleic acids, polypeptides, and cells found in nature.

In one embodiment the E3 ligase complex is of the multi-subunit Cullin-RING E3 ubiquitin ligase type.

In one embodiment the component or domain actually possesses E3 ligase catalytic activity.

In another embodiment the component or domain does not possesses E3 ligase catalytic activity (e.g. is a scaffold component, an adaptor component, a substrate recognition component) but has E3 ligase recruitment activity.

A further aspect of the present invention relates to a nucleic acid molecule encoding a fusion protein as described above. The nucleic acid molecule may be operatively linked to an expression control sequence, e.g. an expression control sequence which allows expression of the nucleic acid molecule in a desired host cell, or one which allows inducible expression. The nucleic acid molecule may be located on a vector, e.g. a plasmid, a bacteriophage, a viral vector, a chromosomal integration vector, lipid nanoparticles etc. or may be “naked” nucleic acid, for example mRNA, which can be delivered via lipid nanoparticles.

A further aspect of the present invention relates to a pharmaceutical composition comprising as an active agent at least one fusion protein as described above.

Also described are uses of, or methods employing, the fusions of the invention in therapy e.g. for treating diseases of protein aggregation.

Some of these aspects and embodiments of the invention will now be described in more detail:

In one embodiment the component or domain comprises or is a RING domain.

Tripartite motif (TRIM) proteins constitute a protein family based on a conserved domain architecture (known as RBCC) that is characterized by a RING (Really Interesting New Gene) finger domain, one or two B-box domains, a Coiled-coil domain, and a variable C-terminus.

In one embodiment the component or domain is derived from a TRIM polypeptide.

In one embodiment the component or domain comprises or is a RING-B-box (see SEQ ID NO. 6).

The optional incorporation of the Box domain may provide an additional layer of control of the monomer/aggregate selectivity owing to its ability to self-associate or bind to the RING.

The replacement of RING domains with heterologous TRIM domains, exchanging them between IM proteins, is known in the art. See Li et al., J. Virol. (2006) 6198-6206. RING domains were described by Freemont et al, Cell. 1991 Feb. 8; 64 (3): 483-4. The domains are believed to function as E3 ligases; see Meroni & Roux, BioEssays 27 (11): 1147-1157 (2005).

RING domains include a specialized type of Zn-finger of 40 to 60 residues that binds two atoms of zinc; defined by the ‘cross-brace’ motif:

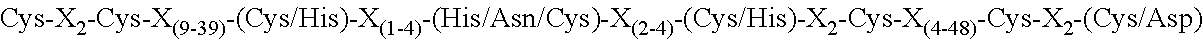

(Deshaies R J et al and Joazeiro C et al, RING Domain E3 Ubiquitin Ligases, Annu. Rev. Biochem (2009) 78:399-434).

There are two variants within the family, the C3HC4-type and a C3H2C3-type (RING-H2 finger), which have a different cysteine/histidine pattern.

C3HC4 has the motif: CiX_iCiX¤fi′f′iCiX¤\hat{ }i′′iHiX¤_i′′iCiX_iCiXi¤aiae ′iCiX_iC

Examples of RING class E3 ubiquitin ligases are given in Balaji, Vishnu, and Thorsten Hoppe cited supra, and are herein individually incorporated by reference.

Preferred RING domains are derived from TRIM proteins. In one embodiment, the RING domain is derived from the group consisting of TRIM5α, TRIM7, TRIM19, TRIM21 and TRIM28.

The RING domain of TRIM21 is responsible for degrading bound antibody/antigen complexes. This is due to the E3 ubiquitin ligase activity of the RING domain.

The sequence of TRIM21 is shown in SEQ ID NO: 1.

RING E3 ligase domains are found in a variety of proteins. Other RING domains include a RING domain from a protein X-linked mammalian inhibitor of apoptosis (XIAP) and a RING domain of DER3/Hrd1. Therefore, the use of RING domains derived from other protein families in the fusion proteins are also encompassed.

Examples of RING domains which can be utilised directly, or modified for use in, the present invention are set out in Table 1 hereinafter. The fusion protein may include the proteins described therein, or the RING containing sequence shown, or a part of, or active variant thereof. Portions of the RING domain will typically be equal to or at least 25, 30, or 35 amino acids long.

As explained above a RING domain for use in the present invention will possess E3 ubiquitin ligase activity i.e. the ability to catalyze covalent transfer of ubiquitin to the RING-containing protein itself or to another protein. This activity is triggered or enhanced in relation to the aggregated protein target by clustering, for example where at least two RING domains brought into proximity.

In one embodiment of the invention the RING domain is derived from amino acids 1-85 of SEQ ID NO: 1 (=SEQ ID NO: 2).

In one embodiment the domain comprises amino acids 3-81 of SEQ ID NO: 2, preferably amino acid residues 1-81 of SEQ ID NO: 2, more preferably the sequence of SEQ ID NO: 2 or a variant thereof.

As explained above, in one embodiment the component or domain does not actually possesses E3 ligase activity per se. It may, for example, serve another purpose within the complex e.g. native substrate recognition or recruitment, or form a structural scaffold.

As demonstrated in the Examples hereinafter, provided that the E3 ligase activity can be recruited to the vicinity of the aggregate, the fusion protein will still be effective.

Thus in one embodiment the component is the VHL (“Von Hippel-Lindau”) domain or protein. VHL is an E3 ubiquitin ligase component that recruits substrates for ubiquitination and subsequent proteasomal degradation.

In one embodiment the VHL polypeptide comprises SEQ ID No. 5 or a variant sharing at least 70% identity therewith.

The aggregation of native proteins such as tau, alpha-synuclein and TDP43 drives neurodegeneration in several common proteopathic diseases. Diseases caused by aggregates of the protein tau are the most numerous class of neurodegenerative diseases, and are characterised by the progressive appearance of tau assemblies throughout the brain over time.

The assembled, disease-associated states can extend by monomer addition into large filaments and macromolecular inclusions such as neurofibrillary tangles (tau) and Lewy bodies (alpha-synuclein).

The molecular architecture of some of these assemblies for tau, α-synuclein and TDP43 have now been resolved by CryoEM, revealing differentially folded but highly ordered fibrillar structures (see e.g. Shi, Y., Zhang, W., Yang, Y. et al. Structure-based classification of tauopathies. Nature 598, 359-363 (2021). https://doi.org/10.1038/s41586-021-03911-7; Yang, Y., Shi, Y., Schweighauser, M. et al. Structures of α-synuclein filaments from human brains with Lewy pathology. Nature (2022). https://doi.org/10.1038/s41586-022-05319-3; Arseni, D., Hasegawa, M., Murzin, A. G. et al. Structure of pathological TDP-43 filaments from ALS with FTLD. Nature 601, 139-143 (2022). https://doi.org/10.1038/s41586-021-04199-3).

As explained above, the present invention utilises a fusion protein having a first portion comprising a sequence of a protein which aggregates pathologically in neurodegenerative disease. This protein targets the pathological protein aggregates and inhibits their formation and/or increases their degradation or clearance.

Those skilled in the art recognise that a common aetiology in these diseases is the progressive accumulation of misfolded insoluble and non-functional protein aggregates (Fink, Anthony L. “Protein aggregation: folding aggregates, inclusion bodies and amyloid.” Folding and design 3.1 (1998): R9-R23). This aggregation may occur via homotypic interactions of corresponding segments of the proteins. These aggregates may be in well-ordered structures, for example fibrils, which may be referred to as “amyloid” (see e.g. Chiti, Fabrizio, and Christopher M. Dobson. “Protein misfolding, amyloid formation, and human disease: a summary of progress over the last decade.” Annual review of biochemistry 86 (2017): 27-68-Table 1). In some cases these insoluble protein precipitates may be more disordered and referred to as “nonamyloid” (see e.g. Chiti, Fabrizio, and Christopher M. Dobson, supra, Table 2).

The fibrillar nature of tau inclusions, and those found in other neurodegenerative diseases²³, provides a consistent characteristic to exploit for therapeutic purposes.

Non-limiting examples of proteins which can be used in the E3 ligase-bait fusions of the present invention, and example diseases in which the proteins aggregate pathologically, include those shown in Table 2:

TABLE 2

| Protein | Diseases |
|---|---|
| Tau protein (3 repeat or 4 repeat isoforms) | Tauopathies with 4R tau assemblies: Corticobasal |
| | degeneration, Argyrophilic grain disease, Progressive |
| | supranuclear palsy (PSP), Frontotemporal lobar degeneration |
| | with tau pathology (FTLD-tau) |
| | Tauopathies with 3R tau assemblies: Pick’s disease, FTLD-tau |
| | Tauopathies with 3R and 4R tau assemblies: Alzheimer’s |
| | disease, Chronic traumatic encephalopathy, FTLD-tau |
| TDP-43 | Amyotrophic lateral sclerosis (ALS), Frontotemporal lobar |
| | degeneration with ubiquitin positive inclusions (FTLD-U) |
| Alpha-synuclein | Parkinson’s disease, Dementia with Lewy Bodies, Multiple |
| | System Atrophy, Alzheimer's disease |
| Huntingtin | Huntington’s disease |
| Rhodopsin | Retinitis pigmentosa (inherited disorders that involve loss of |
| | specialised neuronal cells in the retina) |
| Super Oxide Dismutase | Amyotrophic lateral sclerosis |
| Fused in sarcoma (FUS) protein | Mutations in FUS, including P525L, are associated with the |
| | most aggressive, early-onset forms of ALS as well as rare |
| | forms of FTD |

Example sequences are provided in Table 3 hereinafter.

In one embodiment the protein which aggregates pathologically in neurodegenerative disease is selected from or derived all or part of one of the proteins described in Table 2 or Table 3, or a variant thereof sharing the same biological activity (here: the ability to be incorporated into aggregates of the native protein to form co-aggregates).

In particular, in one embodiment the protein aggregation portion of the fusion protein comprises all or part of sequence shown in Table 3 or of a variant sharing at least 70% identity therewith.

Typically the protein aggregation portion of the fusion protein comprises at least 20, 25, 30, 40 or 50 amino acids. However this portion may be much longer e.g. at least 100, 200, 300, 350, or 400 amino acids. In some embodiments it is less than 400, 450, or 500 amino acids.

As explained in the Examples hereinafter, the E3 ligase-bait system of the invention can be used to target aggregates for clearance, without significant impact on the native (soluble) non-pathogenic activity of these proteins.

The utility of these example proteins in the present invention is discussed in more detail as follows:

### Tau Proteins

4R tauopathies are characterised by deposition of filaments with enrichment for 4R tau isoforms. Familial FTLD-tau can be cased by rare mutations in MAPT, demonstrating a critical involvement of tau (Bonner 2005 PMID 16014652). In progressive supranuclear palsy (PSP), tau has a causative role exemplified by instances of familial variants with MAPT mutations (Morris et al 2002 PMID: 11861703). MAPT H1/H2 haplotype is implicated in disease risk in corticobasal degeneration (CBD) and argyrophylic grain disease (AGD), strongly supporting a causative role of tau in these pathologies (Houlden et al 2001 PMID: 11425937; Conrad et al 2004 PMID: 15030402).

Rare mutations in MAPT such as G389R cause Pick's disease and FTLD-tau with 3R filaments, supporting a causative role of 3R tau isoforms in disease etiology (Murrell et al 1999 PMID: 10604746).

Some tau pathology comprises both 3R and 4R tau isoform, which corresponds closely with disease progression. In AD, pathological tau comprises both 3R and 4R and is closely associated with cognitive decline (Nelson 2013 PMID: 22487856). A similar but non-identical tau fold comprising all six tau isoforms is observed in chronic traumatic encephalopathy (CTE), (Falcon et al 2019 PMID: 30894745). Rare familial mutations such as V337M and R406W can cause FTLD-tau with both 3R and 4R filaments (Goedert et al 2017 PMID: 28772101).

### TDP-43

TAR DNA binding protein 43 (TDP-43) is a 414 amino acid protein found in ALS and FTD-TDP43 inclusions (Arai 2006 PMID: 17084815). Its role as a cause of neurodegeneration is exemplified by mutations in TARDBP that are causative of ALS and FTLD-U (Sreedharan 2008 PMID: 18309045, Neumann et al 2006 PMID: 17023659). Recent structures of TDP43 filamentous core from patients with Type B pathology reveals a core that extends from amino acid 282-360 (Arseni et al 2022, https://doi.org/10.1038/s41586-021-04199-3).

Antibody therapy against TDP43 in vivo appears to work through TRIM21 (PMID: 33021970), supporting the beneficial effects of a E3 ligase-bait strategy.

In some embodiments of the invention, the TDP43 sequence within the E3 ligase-bait may lack the nuclear localisation signal, in order to target cytoplasmic aggregates. In one embodiment the RING domain is present at the C-terminal end of the fusion with the TDP43 sequence.

### Alpha-Synuclein

Alpha-synuclein is a core component of Lewy bodies (found in PD and DLB) and inclusions found in MSA (Spillantini et al 1997 PMID: 9278044; Spillantini et al 1998 PMID: 9726379). A causative role in pathogenesis is exemplified by the fact that SNCA copy number variants and point mutations cause inherited forms of PD (Ibanez 2004 PMID: 15451225; Singleton et al 2003, PMID: 14593171). The filament structure from multiple system atrophy (MSA) has been resolved, and shown to be different to DLB by Schweighauser et al., 2020 (https://doi.org/10.1038/s41586-020-2317-6).

In one embodiment the RING domain is present at the C-terminal end of the fusion with the synuclein sequence.

### Huntingtin (HTT)

It has been reported that expanded CAG repeats encoding poly-glutamine cause Huntington's disease (Lee et al 2019 PMID: 31398342). In one embodiment the RING domain is fused directly or indirectly to a portion of HTT that includes exon 1 or polyglutamine repeats.

### Rhodopsin

Mutations in RHODOPSIN (RHO) lead to retinitis pigmentosa (RP), a rod photoreceptor degeneration that invariably causes vision loss. P23H is the most common mutant and is inherited in an autosomal dominant manner.

In one embodiment the RING domain is present at the C-terminal end of the Rhodopsin carrying the specific causative mutation (e.g. P23H). It has been reported that a CRISPR/Cas9 strategy can be used to specifically inactivate the P23H RHO mutant, while preserving the WT allele in vitro (Giannelli et al 2018 PMID 29281027).

### Super Oxide Dismutase

Mutations in SOD1 can cause Amyotrophic lateral sclerosis (Deng et al., 1993, 10.1126/science.8351519) (Saccon et al., 2013, doi: 10.1093/brain/awt097). Mutations have been found along the length of the protein, and all reduce native SOD1 activity. SOD1 is reported to form ThT positive aggregates (Chattopadhyay et al., 2008 https://www.ncbi.nlm.nih.gov/pmc/articles/PMC2585484/) which can be propagated in mice (Ayers et al., 2014 https://link.springer.com/article/10.1007/s00401-014-1342-7)

Antibody therapy against SOD1 improves lifespan in mice (DOI: 10.1126/scitranslmed.aah3924) and reduces motor neuron toxicity in other models (Benkler et al 2018 PMID: 30401824), indicating supporting the fact that a E3 ligase-bait strategy will be effective against these aggregates.

### FUS

FUS mutations cause ALS (Kwiatkowski Jr et al., 2009, doi: 10.1126/science. 1166066) and FTD (Langenhove et al., 2010, DOI:
https://doi.org/10.1212/WVNL.0b013e3181ccc732).

It has been reported that an ASO mediated knockdown of FUS levels in mutant mice (and a human subject) reduces aggregates and improves lifespan, supporting the use of a E3 ligase-bait approach to achieve the same (https://doi.org/10.1038/s41591-021-01615-z).

As explained above, the two portions of the fusion protein may be linked directly by a single peptide bond or through a peptide linker containing one or more amino acid residues e.g. a flexible linker.

Furthermore the first and second portions can be in any polarity e.g. the protein aggregation domain may be on C-terminal end N-terminal side of the RING domain.

A fusion protein according to the invention may have additional N-terminal and/or C-terminal amino acids or sequences, and/or additional domains (e.g. fluorescent protein domains) at the termini or located between the RING domain and protein aggregation domain.

In other embodiments standard linker sequence known in the art may be also be used, for example polyglycine or polyserine amino acid sequences may be used, or mixed glycine/serine linkers. Preferred linkers are shown within SEQ ID Nos 3 or 4 e.g. GSGGGSG, or linkers based on GSSS e.g. (GSSS)₃.

The linker length can vary in size. In one embodiment the linker sequence between the protein aggregation domain and the RING domain is between 1-200, 1-100, 1-50 amino acid in length, preferably 1-35, 1-30, 1-25, 1-20, 1-15 or 1-10 amino acids in length. More preferably the linker is 1-6 amino acids in length, for example 1, 2, 3, 4, 5, 6, 7, 8, 9 or 10 amino acids in length.

Non-limiting examples of E3 ligase-baits based on a tau protein domain are shown in the SEQ ID No.s 3 and 4. In one embodiment the E3 ligase-bait is one of these or a variant thereof e.g. substantially identical to one of these. In other embodiments the protein aggregation domain is selected according to the target protein or relevant disease (non-limiting examples being given in Tables 3 and 4) arranged in suitable polarity with a RING domain, optionally via one or more linkers.

As explained above fusion proteins of the invention are typically prepared by DNA recombination techniques standard in the art.

For example polynucleotides encoding the fusion proteins may be provided by a process comprising:
(a) providing a first nucleic acid encoding the first portion;
(b) providing a second nucleic acid encoding the second portion;
(c) combining said first and second nucleic acids, for example within a single vector to provide a single ORF.

Generally speaking, those skilled in the art are well able to construct vectors and design protocols for recombinant gene expression. Suitable vectors can be chosen or constructed, containing, in addition to the elements of the invention described above, appropriate regulatory sequences, including promoter sequences, terminator fragments, polyadenylation sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, (1995, and periodic supplements).

The term “expression vector” refers to a DNA construct containing a DNA sequence that encodes the specified polypeptide and is operably linked to a suitable control sequence capable of effecting the expression of the polypeptides in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself.

Expression vectors encoding the fusion proteins of the invention may be for in vitro production of those fusion proteins, or for in vivo gene therapy.

The invention also provides hosts cell comprising such nucleic acid constructs. The invention also provides hosts cell transfected to transformed by such nucleic acid constructs.

The terms “transfect”, “transfection”, “transfected”, and like terms refer to the introduction of a gene into a eukaryotic cell, such as a neuron or keratinocyte, and includes “transduction,” which is viral-mediated gene transfer, for example, by use of recombinant AAV, adenovirus (Ad), retrovirus (e.g., lentivirus), or any other applicable viral-mediated gene transfer platform.

“Transformation” means a transient or permanent genetic change induced in a cell following incorporation of new DNA (i.e., DNA exogenous to the cell). Where the cell is a mammalian cell, a permanent genetic change is generally achieved by introduction of the DNA into the genome of the cell.

The invention also provides a method for preparing fusion proteins of the invention, the method comprising cultivating or maintaining a host cell comprising (or transfected or transformed with) the nucleic construct or vector described above under conditions such that said host cell produces the fusion protein, optionally further comprising isolating the fusion protein.

In one embodiment, the E3 ligase-bait polypeptides (or other agents) of the invention are introduced directly into a cell or the subject to provide their effect or medical benefit. This may be achieved by inhibiting formation of pathological aggregates, or increasing degradation or clearance of pathological aggregates, of an aggregating protein in a cell.

As explained above the fusion proteins are incorporated into a co-aggregate with the aggregating protein, and clustering of the domains (e.g. RING domains) and/or assembly of a complete E3 ligase complex within the co-aggregate causes ubiquitination of the co-aggregate, thereby leading to clearance of the co-aggregate from the cell. The proximity and enhancement achieved by clustering means that normal cytosolic proteins (not present in aggregates) have a reduced likelihood of being degraded by the E3 ligase-bait system.

In one aspect the invention provides a method of therapeutic treatment of an individual (e.g. of a disease or disorder, the terms are used interchangeable) the method comprising administering (directly or indirectly) to the individual a therapeutically-effective amount of a fusion protein of the invention. Relevant diseases are discussed above in Table 2, along with the respect proteins from which the aggregating protein domain can be derived.

Also provided is use of a fusion protein of the invention for treatment by therapy of the human or animal body, or use of a fusion protein of the invention, or other agent described herein, for preparation of a medicament for this purpose.

Thus the specification herein defines methods of treating diseases as discussed herein (e.g. proteopathic diseases) using a fusion protein of the invention, or other agent described herein.

Further disclosed herein is a fusion protein of the invention, or other agent described herein, for use in the treatment of those diseases.

Likewise, further disclosed herein are use of the corresponding agents or combinations of agents in the preparation of a medicament for use in the treatment of those diseases.

Discussion of methods of treatment herein will be understood to apply mutatis mutandis to these uses, and vice versa. Likewise methods of treatment herein will be understood to apply mutatis mutandis to methods used in non-human test animals.

While it is possible for the fusion protein to be used (e.g., administered) alone, it is often preferable to present it as a composition or formulation e.g. with a pharmaceutically acceptable carrier or diluent. This will comprise an effective amount or dose to achieve the intended purpose, when suitably administered to an individual.

The precise nature of the carrier or other material combined with the fusion protein of the invention for pharmaceutical use will depend on the route of administration, which may be by bolus, infusion, injection or any other suitable route, as discussed below.

The determination of an effective dose is well within the capability of trained personnel. For any compounds, the therapeutically effective dose can be estimated initially either in cell culture assays, e.g., of cell lines or in animal models, usually but not exclusively mice. The animal model may also be used to determine the appropriate concentration range and route of administration. Based on such pilot experiments, useful doses and routes for administration in humans can be determined. A therapeutically effective dose refers to that amount of active ingredient, for example fusion protein of the invention, which is sufficient for treating a specific condition (therapeutic efficacy) balanced against any potential toxicity.

Long-acting pharmaceutical compositions may be administered every 3 to 4 days, every week or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art.

Generally, a daily dose of between 0.01 μg/kg of body weight and 100 mg/kg of body weight of agent according to the invention may be used for treating, ameliorating, or preventing disease. More preferably, the daily dose of agent is between 1 mg/kg of body weight and 100 mg/kg of body weight, more preferably between 10 mg/kg and 10 mg/kg body weight, and most preferably between approximately 100 mg/kg and 10 mg/kg body weight.

The duration of treatment may be:
1 to 14, e.g. 1, 2, 3, 4, 5, 6, 7, 8, 9, 10, 11, 12, 13 or 14 days, or
1 to 4, e.g. 1, 2, 3 or 4 weeks, or
1 to 12, e.g. 1, 2, 3, 4, 5, 6, 7, 9, 9, 10, 11, or 12 months.

For prophylaxis, the treatment may be ongoing.

In some embodiments the agent or agents may be administered to a subject or individual during late-stage disease.

In all cases the treatment duration will generally be subject to advice and review of the physician.

In one embodiment, the dosage of the fusion protein may be 20-500 mg twice per week, weekly, every ten days, bi-weekly, every three weeks, or every four weeks.

Most preferably the given dose will be between 50 mg and 200 mg twice per week, weekly, or bi-weekly.

The pharmaceutical compositions detailed in this invention may be administered by any number of routes including, but not limited to, intravenous, intramuscular, intra-arterial, intramedullary, intrathecal, intraventricular, intraocular, transdermal, subcutaneous, intraperitoneal, intranasal, enteral, topical, sublingual or rectal means.

A preferred mode of administration will be parenteral, for example sub-cutaneous or intravenous administration.

For parenteral administration, e.g. by injection, the pharmaceutical composition comprising the fusion protein of the invention may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilisers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

In one embodiment, the E3 ligase-bait polypeptides of the invention are expressed in vivo to provide their medical benefit. This is achieved by use of nucleic acids (polynucleotides) encoding the E3 ligase-bait polypeptide. Typically the polynucleotide is in the form of, or comprised within, a genetic construct comprising an open reading frame encoding the E3 ligase-bait polypeptide under transcriptional control of transcriptional control element.

In one aspect the invention provides a method of therapeutic treatment of an individual (e.g. of a disease or disorder, the terms are used interchangeable) the method comprising administering to the individual a therapeutically-effective amount of a nucleic acid encoding a fusion protein of the invention. Relevant diseases are discussed above.

Also provided is use of a nucleic acid encoding a fusion protein of the invention for treatment by therapy of the human or animal body, or use of a nucleic acid encoding a fusion protein of the invention for preparation of a medicament for this purpose. Discussion of methods of treatment herein will be understood to apply mutatis mutandis to these uses, and vice versa.

Preferably, a nucleic acid construct for use in gene therapy contains a promoter to facilitate expression of the E3 ligase-bait polypeptide-encoding DNA within the target cell.

“Promoter” means a minimal DNA sequence sufficient to direct transcription of a DNA sequence to which it is operably linked. “Promoter” is also meant to encompass those promoter elements sufficient for promoter-dependent gene expression controllable for cell-type specific, tissue specific or inducible by external signals or agents; such elements may be located in the 5′ or 3′ regions of the native gene.

Promoters may be any known in the art suitable for gene therapy-see e.g. Papadakis, E. D., et al. “Promoters and control elements: designing expression cassettes for gene therapy.” Current gene therapy 4.1 (2004): 89-113; and Joshi C R, Labhasetwar V, Ghorpade A. “Destination Brain: the Past, Present, and Future of Therapeutic Gene Delivery.” J Neuroimmune Pharmacol. 2017; 12 (1): 51-83. Promoters may be natural nucleotide sequences, or synthetic combinations of minimal promoter sequences together with other regulatory elements such as enhancers. Examples of commonly used promoters include hSyn, mdl, CBA, Ef1a, TH, CMV, mDIx5/6, DRD2, Drd1a, SSFV, TRE3GS.

Specificity of expression can be achieved by regional and cell-type specific expression of the fusion protein exclusively e.g. using a tissue or region specific promoter.

Alternatively a promoter may be an inducible promoter, which can be activated by an exogenous agent e.g. administered to a subject.

For example the promoter may direct cell-specific expression in neurons e.g. CNS neurons, such as spinal cord cells, or brain cells or nerve cells, or may direct expression in glial cells.

A promoter is “specific” to specified cells if it causes gene expression in those cells of a gene to a sufficient extent for production of useful or therapeutically effective amounts of the described E3 ligase-bait polypeptides in the specified cells, and relatively lower (and non-harmful) expression elsewhere in the context of the use, e.g. therapeutic use.

An example is the Camk2a (alpha CaM kinase II gene) promoter, which drives expression in relatively specifically in the forebrain-see e.g. Sakurada et al (2005) “Neuronal cell type-specific promoter of the alpha CaM kinase II gene is activated by Zic2, a Zic family zinc finger protein.” Neurosci Res. 2005 November; 53 (3): 323-30. Epub 2005 Sep. 12.

Other neuronal cell type-specific promoters include the NSE promoter (Liu H. et al., Journal of Neuroscience. 23 (18): 7143-54, 2003); tyrosine hydroxylase promoter (Kessler M A. et al., Brain Research. Molecular Brain Research. 112 (1-2): 8-23, 2003); myelin basic protein promoter (Kessler M A. et al Biochemical & Biophysical Research Communications. 288 (4): 809-18, 2001); glial fibrillary acidic protein promoter (Nolte C. et al., GLIA. 33 (I): 72-86, 2001); neurofilaments gene (heavy, medium, light) promoters (Yaworsky P J. et al., Journal of Biological Chemistry. 272 (40): 25112-20, 1997) (All of which are herein incorporated by reference at least for the sequence of the promoters and related sequences.) The NSE promoter is disclosed in Peel A L. et al., Gene Therapy. 4 (1): 16-24, 1997) (pTR-NT3myc; Powell Gene Therapy Center, University of Florida, Gainesville FL). A further suitable promoter is the Synapsin1 promoter (see Kügler et al “Human synapsin 1 gene promoter confers highly neuron-specific long-term transgene expression from an adenoviral vector in the adult rat brain depending on the transduced area.” Gene Ther. 2003 February; 10 (4): 337-47). A further suitable promoter is the cd68 promoter, expressed in microglia. Promoters suitable for general expression include the EF1a or CAG promoters.

In one embodiment a vector encoding a E3 ligase-bait polypeptide may comprise any of these promoters.

Vectors may be used to effect permanent transformation, or may be only be transiently expressed in the brain.

Any of a variety of vectors can be used in accordance with the invention to produce E3 ligase-bait polypeptide-expressing cells. A vector for use in the therapies of the present invention will be suitable for in vivo gene therapy protocols. The vector may be a stable integrating vector or a stable non-integrating vector. A preferred vector is viral vector, such as a lentiviral or AAV (Adeno-associated virus) vector.

The use of both these types of viral vector is well known in the art for gene therapy. By way of example only, WO2008011381 describes the use of these and other vectors for expressing polypeptides in a subject. The content of that application, in respect of its description of the preparation and characteristics of AAV and lentiviral vectors is specifically incorporated herein by reference.

Briefly, as described in WO2008011381, AAV is a defective parvovirus and is a preferred vector because it can infect many cell types and is nonpathogenic to humans. AAV type vectors can transport about 4 to 5 kb and wild type AAV is known to stably insert into chromosome 19.

In another type of AAV vector, the AAV contains a pair of inverted terminal repeats (ITRs) which flank at least one cassette containing a promoter which directs cell-specific expression operably linked to a heterologous gene (here: a E3 ligase-bait polypeptide). Further information can be found in U.S. Pat. No. 6,261,834. AAV vectors are discussed in WO2018/175443.

Viral vectors are commercially available e.g. from Viralgen, Parque Cientifico y Tecnológico de Gipuzkoa, Paseo Mikeletegi 83, 20009 San Sebastián, Spain.

Lentiviral vectors are a special type of retroviral vector which are typically characterized by having a long incubation period for infection. Furthermore, lentiviral vectors can infect non-dividing cells. Lentiviral vectors are based on the nucleic acid backbone of a virus from the lentiviral family of viruses. Typically, a lentiviral vector contains the 5′ and 3′ LTR regions of a lentivirus, such as SIV and HIV. Lentiviral vectors also typically contain the Rev Responsive Element (RRE) of a lentivirus, such as SIV and HIV. Examples of lentiviral vectors include those of Dull, T. et al., “A Third-generation lentivirus vector with a conditional packaging system” J. Virol 72 (11): 8463-71 (1998).

Thus in one embodiment there is provided an expression vector comprising the polynucleotide of the invention described above. The vector may be a viral vector e.g. an adenovirus vector and/or an adeno-associated vector (AAV), which is optionally selected from the group consisting of: AAV1, AAV2, AAV3, AAV4, AAV5, AAV6, AAV7, AAV8, AAV9, AAV10, AAV11, AAV 12, and hybrids thereof.

One preferred AAV is AAV-9P31, which is a derivative of AAV9 (see WO2020072683A1, and PMID: 33553485).

AAV vectors are known in the art which are capable of crossing the blood brain barrier (see e.g. Nonnenmacher, Mathieu, et al. “Rapid evolution of blood-brain-barrier-penetrating AAV capsids by RNA-driven biopanning.” Molecular Therapy-Methods & Clinical Development 20 (2021): 366-378) and such may be preferred. Examples include AAV-F.

Alternatively, the vector may be a herpes virus vector, a retrovirus vector, or a lentivirus vector.

In methods of delivering vectors or nucleic acids encoding E3 ligase-bait polypeptides according to any aspect described herein to a cell or to a patient, the vector or nucleic acid may be delivered by any useful method, in any useful form, as is recognized by those of ordinary skill in the field of genetic therapies.

For example liposomes or nanoparticles comprising the nucleic acid may be injected at a desired site, such as in or adjacent to specific neuronal tissue. In other aspects, a recombinant viral particle (transducing particle), is delivered, for example, injected, at a desired site, such as in or adjacent to, or otherwise targeting specific neuronal tissue. The nucleic acid may be injected once or more than once in order to establish sufficient expression of the E3 ligase-bait polypeptide in the target neuron.

In particular, delivery can be via direct injection into the brain using known methodologies, such as direct interstitial infusion, burr-hole craniotomy and stereotactic injection (see e.g. “Stereotactic and Functional Neurosurgery” Editors: Nikkhah & Pinsker; Acta Neurochirurgica Supplement Volume 117, 2013).

AAV may be used advantageously for intraocular gene administration (see e.g. Ail, Divya, et al. “Systemic and local immune responses to intraocular AAV vector administration in non-human primates.” Molecular Therapy-Methods & Clinical Development 24 (2022): 306-316).

As described in WO2008096268, in gene therapy embodiments employing viral delivery of the E3 ligase-bait polypeptide, the unit dose may be calculated in terms of the dose of viral particles being administered. Viral doses include a particular number of virus particles or plaque forming units (pfu). For embodiments involving AAV, particular unit doses include 10³, 10⁴, 10⁵, 10⁶, 10⁷, 10⁸, 10⁹, 10¹⁰, 10¹¹, 10¹², 10¹³ or 10¹⁴ pfu or vector genomes. Particle doses may be somewhat higher (10 to 100 fold) due to the presence of infection-defective particles.

In one embodiment a vector is injected as 500 microL of a suspension of 5×10¹¹ vg/ml (=2.5×10¹¹ viral genomes).

The agents utilised in the present invention may be provided as a composition e.g. a “pharmaceutical composition” (e.g., formulation, preparation, medicament) comprising at least one agent of the invention (i.e. fusion protein, or nucleic acid encoding it, or relevant vector or capsid or particle) described herein, and a pharmaceutically acceptable carrier, diluent, or excipient.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition comprising at least one agent of the invention, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

In some embodiments the pharmaceutical composition comprises, or consists essentially of, or consists of, the agent of the invention, and a pharmaceutically acceptable carrier, diluent, or excipient. In some embodiments, the composition further comprises other active agents, for example, other therapeutic or prophylactic agents.

In some embodiments the methods or treatments of the present invention may be combined with other therapies, whether symptomatic or disease modifying e.g. a second therapeutic agent believed to show therapeutic benefit in the relevant cancers. The term “treatment” includes combination treatments and therapies, in which two or more treatments or therapies are combined, for example, sequentially or simultaneously. For example it may be beneficial to combine treatment with a fusion protein of the invention as described herein with one or more other (e.g., 1, 2, 3, 4) agents or therapies.

Appropriate examples of co-therapeutics will be known to those skilled in the art on the basis of the disclosure herein. Typically the co-therapeutic may be any known in the art which it is believed may give therapeutic effect in treating the relevant disease.

Wherever amino acid and nucleic acid sequences are discussed herein (for example in respect of encoding fusion proteins or portions thereof), it will be appreciated by the skilled technician that functional variants of the amino acid, and hence encoding nucleic acid sequences, disclosed herein, are also envisaged. Typically such variants will be “substantially identical” to the reference sequence disclosed herein. Typically they will share the biological activity of the reference sequence.

For example the biological activity of the RING domain is the ability to catalyze covalent transfer of ubiquitin to the RING-containing protein itself or to another protein, a property that may be enhanced when at least two RING domains are brought into proximity following assembly into a multimeric complex. The biological activity of the protein aggregation portion is the ability to be incorporated into aggregates of the native protein, to form multimeric co-aggregates.

“Substantially identical” in the context of at least two nucleic acids or polypeptides means that a polynucleotide or polypeptide comprises either a sequence that has at least about 60%, 65%, 70%, 75%, 80%, 85%, 90%, 91%, 92%, 93%, 94%, 95%, 96%, 97%, 98%, or 99% identity to a parent or reference sequence, or any other sequence that includes amino acid substitutions, insertions, deletions, or modifications made only to circumvent the present description without adding functionality.

Calculation of percentage identities between different amino acid/polypeptide/nucleic acid sequences may be carried out as follows. A multiple alignment is first generated by the ClustalX program (pair wise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof.

Alternatively, a substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention.

Due to the degeneracy of the genetic code, it is clear that any nucleic acid sequence could be varied or changed without substantially affecting the sequence of the protein encoded thereby, to provide a functional variant thereof. Degeneratively equivalent (e.g. codon optimised) nucleotide sequences to any of those described herein may of course be used in their place.

Thus suitable nucleotide variants include those having a sequence altered by the substitution of different codons that encode the same amino acid within the sequence, thus producing a silent change. Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid.

It will be appreciated that fusion proteins used or provided according to the invention may be derivatives of native or original sequences, and thus include derivatives that increase the effectiveness or half-life of the agent in vivo. Examples of derivatives capable of increasing the half-life of polypeptides according to the invention include peptoid derivatives, D-amino acid derivatives and peptide-peptoid hybrids.

Fusion proteins according to the present invention may be subject to degradation by a number of means (such as protease activity at a target site). Such degradation may limit their bioavailability and hence therapeutic utility. There are a number of well-established techniques by which peptide derivatives that have enhanced stability in biological contexts can be designed and produced. Such peptide derivatives may have improved bioavailability as a result of increased resistance to protease-mediated degradation. Preferably, a derivative suitable for use according to the invention is more protease-resistant than the protein or peptide from which it is derived. Protease-resistance of a peptide derivative and the protein or peptide from which it is derived may be evaluated by means of well-known protein degradation assays. The relative values of protease resistance for the peptide derivative and peptide may then be compared.

Peptoid derivatives of fusion proteins according to the invention may be readily designed from knowledge of the structure of the active portions. Commercially available software may be used to develop peptoid derivatives according to well-established protocols.

Retropeptoids, (in which all amino acids are replaced by peptoid residues in reversed order) are also able to mimic proteins or peptides according to the invention. A retropeptoid is expected to bind in the opposite direction in the ligand-binding groove, as compared to a peptide or peptoid-peptide hybrid containing one peptoid residue. As a result, the side chains of the peptoid residues are able to point in the same direction as the side chains in the original peptide.

A further embodiment of a modified form of peptides or proteins according to the invention comprises D-amino acid forms. In this case, the order of the amino acid residues is reversed. The preparation of peptides using D-amino acids rather than L-amino acids greatly decreases any unwanted breakdown of such derivative by normal metabolic processes, decreasing the amounts of the derivative which needs to be administered, along with the frequency of its administration.

Other modifications in protein sequences are also envisaged and within the scope of the claimed invention, i.e. those which occur during or after translation, e.g. by acetylation, amidation, carboxylation, phosphorylation, proteolytic cleavage or linkage to a ligand.

The term “derived from” encompasses the terms “originated from,” “obtained from,” “obtainable from,” “isolated from,” and “created from” and generally indicates that one specified material find its origin in another specified material or has features that can be described with reference to the another specified material (which may be termed “reference” or “parent”). The E3 ligase-bait polypeptides herein may be derived from reference or parent sequences, for example form wild type-type aggregating protein sequences and TRIM proteins.

The terms “polynucleotide” encompasses DNA, RNA, heteroduplexes, and synthetic molecules capable of encoding a polypeptide. Nucleic acids may be single-stranded or double-stranded, and may have chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence. Unless otherwise indicated, nucleic acid sequences are presented in a 5′-to-3′ orientation.

As used herein, a “polypeptide” refers to a molecule comprising a plurality of amino acids linked through peptide bonds. The terms “polypeptide,” “peptide,” and “protein” are used interchangeably. Proteins may optionally be modified (e.g., glycosylated, phosphorylated, acylated, farnesylated, prenylated, and sulfonated) to add functionality. The conventional one-letter or three-letter codes for amino acid residues are used, with amino acid sequences being presented in the standard amino-to-carboxy terminal orientation (i.e., N→C).

The terms “Subject”, “individual” or “patient” (the terms are used interchangeably unless context demands otherwise) to be treated will typically refer to a mammal e.g. a human or non-human mammal. Thus in one embodiment the individual is a human subject e.g. a patient. Alternatively the mammal may be a non-human mammal e.g. a test animal such as a rodent (e.g. mouse, rat) or primate. Non-human subjects include rabbit, pig, monkey, chimpanzee, cat, dog, horse, goat, guinea pig. The non-human mammal may be a transgenic mammal. Alternatively the subject or organism may be a bird, fish, reptile or amphibian.

The term “therapeutically-effective amount,” as used herein, pertains to that amount of a compound of the invention, or a material, composition or dosage from comprising said compound, which is effective for producing some desired therapeutic effect, commensurate with a reasonable benefit/risk ratio, when administered in accordance with a desired treatment regimen.

“Therapeutic efficacy” and “toxicity” may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as LD50/ED50. Pharmaceutical compositions, which exhibit large therapeutic indices, are preferred. The dosage is preferably within a range of circulating concentrations that include the ED50 with little or no toxicity. The dosage varies within this range depending upon the dosage employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress (prolonged survival), a halt in the rate of progress, regression of the condition, amelioration of the condition, and cure of the condition. The invention also embraces treatment as a prophylactic measure is also included and “treating” will be understood accordingly. “Prophylaxis” may utilise a “prophylactically effective amount,” which, where used herein, pertains to that amount of an agent which is effective for producing some desired prophylactic effect, commensurate with a reasonable benefit/risk ratio, when administered in accordance with a desired treatment regimen. “Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

As used herein, the terms “wild-type”, “native”, or “reference” refer to polypeptides or polynucleotides that are found in nature. The terms, with respect to a polypeptide, refer to a naturally-occurring polypeptide that does not include a man-made substitution, insertion, or deletion at one or more amino acid positions. The terms with respect to a polynucleotide, refer to a naturally-occurring polynucleotide that does not include a man-made substitution, insertion, or deletion at one or more nucleosides. However, note that a polynucleotide encoding a wild-type or native or reference polypeptide is not limited to a naturally-occurring polynucleotide, and encompasses any polynucleotide encoding that polypeptide.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

The phrase “selected from the group comprising” may be substituted with the phrase “selected from the group consisting of” and vice versa, wherever they occur herein.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

### FIGURES

FIG. 1: Tau-RING prevents seeded aggregation and removes existing aggregates (a) Schematic and representative images of HEK293 reporter cell line expressing tau-venus (TV cells)+/−Tau-RING, seeded+/−10 nM tau aggregates with LF2000. White arrows denote examples of aggregates. (b) Quantification of cells treated as in (a). N=3. (c) Western blot of cells expressing tau-venus+/−Tau-RING, +/−10 nM tau aggregates delivered with LF2000. Blot probed for tau and loading control CypB. (d) Quantification of tau-venus observed in western blot in (c), normalised to CypB. N=3. (e) Quantification of Tau-RING observed in western blot in (c), normalised to CypB. N=3. (f) Western blot of cells expressing tau-venus+/−Tau-RING, +/−10 nM tau aggregates delivered with LF2000. Blot probed for AT8 and loading control CypB. (g) Quantification of AT8 positive tau-venus observed in western blot in (f), normalised to CypB. N=3. (h) TEM of P301S tau or P301S Tau-RING aggregates. (i) HEK293 reporter cell line expressing tau-venus, constitutively bearing tau aggregates (TVA), was infected with lentivirus containing Tau-RING and evaluated over 3 days. (j) Time course of tau aggregates in TVA reporter cells with and without Tau-RING. N=3. (k) Quantification of the number of tau aggregates in TVA cells 72 hours post infection with Tau-RING lentivirus. N=3. Statistical significance for (b) determined by two-way ANOVA and Sidak's multiple comparisons test. Statistical significance for (d), (e), (g) determined by one-way ANOVA and Tukey's multiple comparisons test. Statistical significance for (j), (k) determined by unpaired t-test with Welch's correction. ***, p<0.001. ****, p<0.0001. ns, non-significant.

FIG. 2: Tau-RING co-localises with aggregates and initiates complete degradation, removing seed competent species

(a) BHK cells expressing tau-venus were infected with doxycycline inducible Tau-mCherry-RING. Upon seeding with preformed tau aggregates, tau-venus and Tau-mCherry-RING were observed to co-localise. (b) Quantification of co-localisation of tau-venus and Tau-mCherry-RING. (c) Time-course of Tau-mCherry-RING causing the degradation of tau-venus positive aggregates in a BHK cell. (d) Schematic of the secondary seeding assay. TVA cells were infected with a lentivirus carrying Tau-mCherry-RING. The cells were then sorted for infected cells, and then a pure population of cells expressing tau-venus and Tau-mCherry-RING was cultured for 2 weeks. 1 million cells were collected, lysed, and applied to the HEK293 reporter cell line expressing tau-venus to assess the amount of seed competent species present in the lysate. Lysate was also taken from uninfected TVA cells and TV cells. (e) Representative images of secondary seeding from TVA, TVA+Tau-mCherry-RING and TV cell lysate (f) Quantification of secondary seeding from TVA, TVA+Tau-mCher-ry-RING and TV cells in reporter TV cells. Statistical significance for (f) determined by one-way ANOVA and Tukey's multiple comparisons test. ****, p<0.0001.

FIG. 3: RING dimerization and VCP recruitment are essential to the function of Tau-RING

(a) Schematic of RING dimerization between two TRIM21 dimers. The M72 residue is marked in orange. The M72E muta-tion inhibits RING dimerization. (b) Western blot of cells expressing tau-venus+Tau-RING or Tau-RING-M72E, run on the same gel. (c) TV cells+/−Tau-RING or Tau-RING-M72E, +/−10 nM tau aggregates delivered with Lipofectamine. N=3, n=9. (d) TVA cells+/−lentivirus expressing Tau-RING or Tau-RING-M72E. Aggregates analysed at 72 hours post infection. (e) Diagram of neuronal tau entry assay. P301S 0N4R tau, Tau-RING or Tau-RING-M72E protein was produced with a HiBiT tag, and aggregated. Primary neurons were infected with AAV to express LgBiT, and then aggregated tau species were incubated with the neurons. The amount of tau in the cytosol was quantified by luminescent signal. Once Tau-RING comes into contact with the cytosol it is available for proteasomal degradation, enabling real-time visualisation of degradation. (f) Aggregation of tau-HiBiT, Tau-RING-HiBIT and Tau-RING-M72E-HiBiT protein in the presence of heparin, visualised with Thioflavin T (ThT).

(g) Neuronal entry assay of tau-HiBIT, Tau-RING-HiBIT and Tau-RING-M72E-HiBIT aggregates to the cytosol using a HiBiT-LgBiT split luciferase assay. N=3. (h) Representative western blot of TV cells+/−Tau-RING with the addition of NTC or VCP siRNA after 48 hours, probed for VCP and loading control GAPDH. (i) Quantification of TV cells+/−Tau-RING, seeded with 30 nM tau aggregates using lipofectame, and assessed at 24 hours. Cells were untreated or treated with NTC or VCP siRNA for 48 hours before seeding. N=3, n=9. (j) TV+Tau-RING data from graph (i) plotted on a linear scale to visualise the increase in seeding mediated by VCP siRNA knockdown. N=3, n=9. Statistical significance for (c), (i), (j) determined by two-way ANOVA and Sidak's multiple comparisons test. Statistical significance for (d), (g) determined by one-way ANOVA and Tukey's multiple comparisons test. **, p<0.01, ***, p<0.001. ****, p<0.0001. ns, non-significant.

FIG. 4: Tau-RING is effective with different sized linkers.

a) HEK293 cells expressing tau-venus and tau-CFP-RING are positive for both venus and CFP by live cell imaging. b) Western blot of cells expressing tau-venus+/−Tau-RING or Tau-CFP-RING, probed for tau and loading control CypB. c) Quantification of Tau-RING and Tau-CFP-RING levels in western blot in (b). Statistical significance determined by Unpaired t-test. d) Quantification of tau-venus levels in western blot in (b). Statistical significance determined by one-way ANOVA and Tukeys multiple comparisons test. e) Quantification of HEK293 cells expressing tau-venus+/−Tau-CFP-RING, seeded with 10 nM tau aggregates and analysed at 72 hours. N=3, n=9. Statistical significance determined by two-way ANOVA and Tukeys multiple comparisons test. f) Quantification of HEK293 cells expressing tau-venus+/−Tau-RING, seeded with 10 nM tau aggregates and analysed at 72 hours. N=3, n=9. Statistical significance determined by two-way ANOVA and Tukeys multiple comparisons test. ****, p<0.0001. ns, non-significant.

FIG. 5: Tau-RING reduces seeded aggregation in primary neurons

(a) Schematic of the Venus-P2A-Tau-RING construct, packaged in an AAV vector. Primary neurons were infected on day 2, and full expression of the construct was observed by day 6. (b) Western blot of primary neurons from P301S mice, infected with venus AAV, venus-P2A-tau-RING AAV or untreated. Blot probed for tau, venus and loading control CypB. (c) Quantification of endogenous tau levels in P301S primary neurons+/−infection with venus or venus-P2A-tau-RING AAV. (d) Primary neurons infected with venus or venus-P2A-tau-RING AAV, seeded with 100 nM P301S tau aggregates at day 7. Cells were fixed at day 14. (e) AT8 positive puncta at day 14 in neurons treated as in (d). (f) AT8 positive neuronal cell bodies at day 14 in neurons treated as in (d). (g) Neuronal viability upon seeding with tau aggregates, determined by NeuN count at day 14 in neurons treated as in (d). Statistical significance for (e), (f) determined by two-way ANOVA and Sidak's multiple comparisons test. Statistical significance for (c), (g) determined by one-way ANOVA and Tukey's multiple comparisons test. ****, p<0.0001. ns, non-significant.

FIG. 6: Tau-RING removes tau aggregates in vivo

(a) 4 month old P301S were injected with 1×10¹¹ vgs of venus or venus-P2A-Tau-RING AAV 9P31. No adverse side effects were observed during the duration of treatment, exemplified by steady weight over a 2 month period. (b) Sarkosyl Insoluble (SI) extraction from 1 hemisphere of each mouse. Total tau and SI tau probed for with pan-tau antibody A0024 (Dako). (c) Quantification of SI tau, normalised to total tau present in N=6 mice per group. (d) Representative images from the frontal cortex, showing the co-localisation of the AAV and tau aggregates, visualised by venus fluorophore and stained for with AT8 respectively. (e) Quantification of venus+AT8 positive neurons in the frontal cortex. N=6 mice per group. (f) Quantification of neuronal nuclei, as a measure of toxicity, in the frontal corte. N=6 mice per group. (g) 5 month P301S mice were stereotaxically injected with 5×10¹⁰ vgs of venus-P2A-Tau-RING or venus only AAV PHP.eB in the frontal cortex, and culled one month later. Coronal sections were sliced and evaluated for AT8 positivity. White arrow denotes injected hemisphere. Quantification of AT8 positive, venus positive areas. AT8 positivity was normalised to the uninjected hemisphere of each mouse individually. 3 sections analysed per mouse. N=5 mice. Statistical significance for (c), (e), (f) determined by unpaired t-test with Welch's correction. Statistical significance for (h) determined by nested t-test. **, p<0.01, ****, p<0.0001. ns, non-significant.

FIG. 7: Tau fusions to other RING constructs or VHL E3 ligase removes existing intracellular tau aggregates

a) Quantification of the number of tau aggregates in TVA cells 72 hours post infection with lentivirus coding for the indicated genes. b) Quantification of the number of tau aggregates in HEK293 reporter cell line expressing tau-venus (TV cells)+/−Tau-VHL, seeded+/−30 nM tau aggregates with LF2000. Statistical significance determined by one-way ANOVA and Tukey's multiple comparisons test. ***, p<0.001. c) Western blot of cells expressing tau-venus+/−Tau-VHL. Blot probed for GFP to detect tau-venus and loading control CoxIV. d) Quantification of western blot showed in c).

FIG. 8: a, Schematic of RING domain activation via dimerisation. The 118R and M72E mutations are highlighted in green and purple respectively. b, Time course of Tau-RING+/−118R, M72E, 118R/M72E RING mutations in TVA cells. c, Quantification of the number of aggregates in TVA cells 72 hrs post infection with tau-RING lentivirus. N=3. d, Schematic of inhibitors of degradation pathway components utilised by TRIM21. TAK-243 is an E1 inhibitor, NMS-873 is a VCP inhibitor and MG-132 is a proteasome inhibitor. e, Time-course of TVA cells treated with tau-RING+/−TAK-243, and quantification of end point at 48 hrs post infection. N=3. f, Time course of TVA cells treated with tau-RING+/−NMS-873, and quantification at 48 hrs post infection. N=3. g, Time course of TVA cells treated with tau-RING+/−MG-132, and quantification at 48 hrs. N=3. Statistical significance for c,e, f,g determined by one-way ANOVA and Tukey's multiple comparisons test. **, p<0.01, ***, p<0.001. ****, p<0.0001. ns, non-significant.

FIG. 9: a, Schematic of the TVA cell assay, with a lentivirus used to express different isoforms of tau-RING as examples of different ‘Baits’. Incorporation of the ‘Bait’ into the aggregate leads to proteasomal degradation, and a reduction in the number of puncta by high content microscopy. b, Time course of lentivirus carrying P301S 0N4R tau-RING, WT 0N3R tau-RING or WT 0N4R tau-RING applied to TVA cells. c, Quantification of the number of aggregates in TVA cells treated as in b, 72 hrs post infection. N=3.

d, Quantification of the number of aggregates in HEK293T cells expressing venus-3R tau, seeded with AD derived tau aggregates, +/−0N3R tau-RING. N=3. e, Representative images from cells treated as in d. White arrows denote tau aggregates. f, Quantification of the number of aggregated in HEK293T cells expressing venus-4R tau, seeded with PSP derived tau aggregates, +/−0N4R tau-RING. N=3. g, Representative images from cells treated as in f. White arrows denote tau aggregates. Statistical significance for c determined by one-way ANOVA and Tukey's multiple comparisons test. Statistical significance for d,f, determined by unpaired t-test . . . ****, p<0.0001. ns, non-significant.

FIG. 10: a, Schematic of intravenous injection of P301S mice at 4 months with AAV 9P31. At 6 months, one half of the brain was homogenised in order to extract Sarkosyl Insoluble (SI) tau assemblies and the other half was fixed and analysed for AT8 positive tau aggregates by immunofluorescent staining. b, Representative immunoflourescence images of mice infected with AAV 9P31 hSyn: VPTR or hSyn: VPTR 118R/M72E at 4 months, or injected with PBS, and evaluated at 6 months for tau aggregates via AT8 staining. Venus fluorescence detected from virally expressed protein. Neuronal nuclei were probed for using an antibody against NeuN. Enlarged cortical region shown to exemplify tau aggregate levels. c, Quantification of AT8 positive tau aggregates in frontal cortex, as shown in b. d, Western blot of the sarkosyl insoluble (SI) fraction of mouse brains treated as in b, stained for total human tau (HT7) and hyperphosphorylated tau at serine 422 (pS422). Mouse brain homogenate was also stained with HT7, and probed for Venus protein, in addition to GAPDH as a loading control. e, Quantification of SI HT7. f, Quantification of SI pS422. g, Quantification of homogenate Venus protein levels, from viral infection. h, Footsteps of the median mouse from each condition (VPTR, VPTR 118R/M72E, PBS) on the MouseWalker apparatus. i, Quantification of the time to traverse the walkway for mice from each condition (VPTR, VPTR|18R/M72E, PBS) from 4 to 6 months. N=6-8 mice per group. Statistical significance for c,e,f,g,i determined by unpaired t-test. *, p<0.05. **, p<0.01. ****, p<0.0001. ns, non-significant.

FIG. 11: a,b Quantification of higher HT7 bands in homogenate Western blot from FIG. 10c. Quantification of main HT7 band in homogenate Western blot from FIG. 10c. c, Western blot of mice treated as in FIG. 10, probed for neuron specific β-III-tubulin and loading control GAPDH. d, Quantification of β-III-tubulin levels in c. e, Mice infected with hSyn: Venus AAV 9P31.

FIG. 12: a, Analysis of the frames per video in which feet were in contact with the platform, at 4 months before treatment and at 6 months after treatment. b, Time for mice to cross the MouseWalker platform at 4 and 6 months. c, Number of front paw steps to cross the MouseWalker platform at 4 and 6 months. d, Number of back paw steps to cross the MouseWalker platform at 4 and 6 months. Statistical significance for a,b,c,d, determined by two-way ANOVA and Sidak's multiple comparisons test.

FIG. 13: a) Representative images of HEK293 cells co-transfected with mGreenLantern tagged P525L FUS along with RING-525L FUS-T2A-mCherry.

Doxycycline was used to drive expression of the RING-bait version. The P525L variant of FUS forms cytosolic aggregates that are degraded upon induction of the RING-FUS P525L construct. b) Quantification of cytosolic aggregates 24 hours post after addition of dox to drive expression of RING-bait or a RING only control. N=3. Statistical significance determined by 2way ANOVA.

### METHODS FOR EXAMPLES

### HEK Cell Line Generation

Tau-RING was cloned into lentiviral plasmid smppv2 and lentivirus was generated by transfecting spmmv2 with helper plasmids into HEK293 cells. Virus was harvested after 3 days, filtered, and titrated onto HEK293 cells already expressing tau-venus (TV cells) (McEwan et al., 2017). Stably expressing cells were selected for with puromycin for >3 passages. Colonies were expanded and pooled for use in tau seeding assays. Tau-venus cells with aggregates (TVA cells) were generated by seeding these cells with preformed tau aggregates, and stably propagating the cells which constitutively bore aggregates.

### HEK293 Tau Seeding Assay

HEK293 cells expressing tau-venus+/−Tau-RING were plated at 20,000 cells per well in a black 96 well plate, in 50 μl reduced serum OptiMEM. Pre-formed P301S tau aggregates (aggregated with heparin) were diluted in an equal volume of OptiMEM and mixed with transfection reagent Lipofectamine 2000. After incubating for 20 minutes, 50 μl tau aggregates were added to each well, and incubated for 1 hour. The reaction was neutralised after 1 hour with 100 μl complete DMEM. Plates were then moved to an Incucyte for live cell imaging every 2 hours, for 72 hours. The number of tau aggregates was analysed at 72 hours.

### Neuron Tau Seeding Assay

Primary neurons were generated from P1/2 P301S mice and plated in black 96 well plates. Cultures were infected with AAV PHP.eB expressing Venus or Venus-P2A-Tau-RING under the CAG promoter at day 2. At day 7 pre-formed P301S tau aggregates were added to the media, to a final concentration of 100 nM. Cultures were fixed at day 14 and stained for neuronal nuclei marker NeuN to obtain a neuronal cell count, and AT8 (phospho-tau) to obtain an aggregate count.

### Removal of Endogenous Aggregates

Cell line TVA, which constitutively bears tau aggregates, was plated in 96 well format, and lentivirus expressing P301S Tau-RING was applied to the cells. The number of aggregates was evaluated at 72 hours.

### Secondary Seeding Assay

TVA cells treated+/−tau-RING lentivirus were lysed and total cell homogenate was used as ‘seeding’ material. 1 μl homogenate was applied to new TV cells using Lipofectamine, as described in the ‘HEK293 tau seeding assay’ section. The number of aggregates in TV cells was quantified after 72 hrs.

### WT Tau Seeding Assays

0N3R venus-tau was expressed in HEK293T cells using lentiviral transduction. Tau aggregates derived from Alzheimer's disease (AD) brains were applied to the cells as described in the ‘HEK293 tau seeding assay’ section. Lentivirus encoding 0N3R tau-RING was applied to the cells and the number of aggregates was quantified after 72 hrs. The same assay was carried out with a 0N4R system to test RING-bait against progressive supranuclear palsy (PSP) brain derived aggregates.

### Quantification of Aggregates

Aggregates from both the HEK293 assay and Neuron assay were quantified using Fiji plugin comdet and normalised to cell count.

### In Vivo Experiments

5 month-old mice were stereotaxically injected in one hemisphere of the frontal cortex with 5×10¹⁰ vgs of either Venus or Venus-P2A-Tau-RING AAV PHP.eB in a volume of 1.5 μl. Mice were culled 1 month later, and perfused with 4% PFA. Brains were dehydrated in 30% sucrose, and sliced into 30 μm thick sections using a vibratome. Venus positive layers were selected for analysis of tau aggregates by AT8 staining. Alternatively, 4 month old mice were injected in the tail vein with Venus or Venus-P2A-Tau-RING AAV 9P31 with 1×10¹¹ vgs, or 4×10¹¹ vgs, and culled at 6 months. 1 hemisphere was fixed in 4% PFA for 48 hours, and then prepared for staining as previously described. 1 hemisphere was used for the extraction of sarkosyl insoluble tau, which was analysed by western blot.

### MouseWalker Phenotypic Testing

Mice were videoed walking along the MouseWalker platform, every two weeks from 4-6 months of age. Videos were analysed using a neural network trained using DeepLabCut software.

### EXAMPLES

### Example 1—E3 Ligase-Bait Efficiently Degrades Aggregates Whilst Sparing Soluble Protein

To demonstrate that a “E3 ligase-bait” strategy could be successfully applied to degrade aggregated proteins, we selected tau as both the target of degradation and the bait. For demonstration purposes we used the P301S mutation which causes early onset familial dementia and is pro-aggregant, increasing the rate of tau fibril extension by about 50-fold compared to wildtype tau¹⁰.

For the bait, we fused the TRIM21 RING domain to the C-terminus of 0N4R P301S tau, referred to as Tau-RING. We co-expressed the Tau-RING alongside the target protein, P301S 0N4R tau-venus. This construct has previously been shown to respond to the presence of exogenous tau assemblies by forming aggregates, which present as bright puncta that can be quantified by high-content fluorescence microscopy 8. HEK293 cells expressing P301S 0N4R tau-venus are henceforth referred to as ‘TV cells’

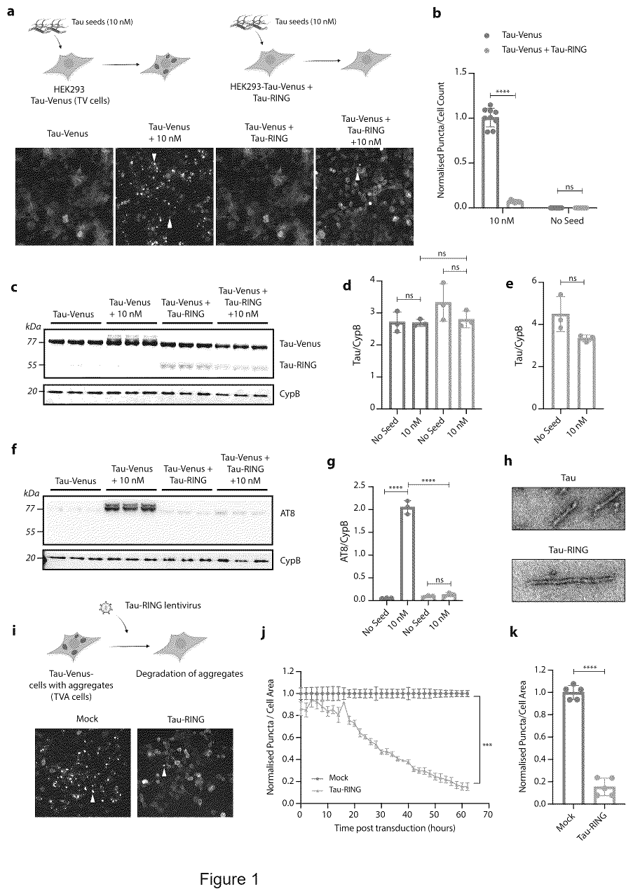

We first tested whether Tau-RING was capable of preventing the de novo formation of new tau aggregates induced by the addition of exogenous misfolded tau seeds. Recombinant 0N4R P301S tau was aggregated with heparin and transfected into the cells to seed aggregation of intracellular tau-venus in TV cells. The resulting tau-venus aggregates were quantified by high content microscopy after 72 hrs. The Tau-RING construct reduced seeded aggregation of the target, P301S tau-venus, by <95% compared to cells that did not express tau-RING (FIG. 1a,b). Tau-RING is therefore capable of inhibiting the seeded aggregation of tau.

To investigate if Tau-RING is acting specifically on nascent tau aggregates or is simply reducing the available pool of cellular tau, we analysed the total levels of tau-venus upon co-expression with Tau-RING (FIG. 1c). No reduction in soluble tau-venus occurred in the presence of Tau-RING, suggesting the construct was not capable of degrading monomeric forms of tau consistent with the clustering mechanism of activation of the TRIM21 RING domain (FIG. 1d). The reduction of Tau-RING and tau-venus upon the addition of aggregates was also non-significant, indicating Tau-RING is highly specific at degrading aggregated species when activated (FIG. 1d,e). A stark reduction in levels of staining by AT8, an antibody that detects tau phosphorylated at the S202/T205 site, was observed upon the addition of tau aggregates in the presence of Tau-RING (FIG. 1f,g). AT8 phosphorylation of Tau-RING was not detectable, consistent with assembled versions of this protein being rapidly degraded before phosphorylation. Tau-RING protein readily formed aggregated species, visualized by TEM. Tau-RING therefore does not inhibit seeded aggregation by preventing the formation of fibrillar aggregates (FIG. 1h). Having demonstrated that tau-RING can degrade tau aggregates arising from seeded aggregation, we considered whether it could deplete pre-formed cytoplasmic tau aggregates. HEK293 P301S tau-venus cells were seeded with aggregates such that they constitutively expressed P301S tau-venus aggregates. This cell line is henceforth referred to as ‘TVA cells’. These cells were infected with a lentivirus coding for tau-RING and monitored for the presence of tau-venus puncta over time (FIG. 1i). Following transduction, the proportion of cells containing tau-venus positive aggregates reduced over 72 hours until only 10% of cells contained tau-venus in the aggregated state (FIG. 1j, k). We hypothesis that as the extension of tau fibrils occurs during both seeded and cell autonomous aggregation, this shared mechanism of incorporation of tau-RING is the mechanism behind the successful degradation of new and existing intracellular fibrils.

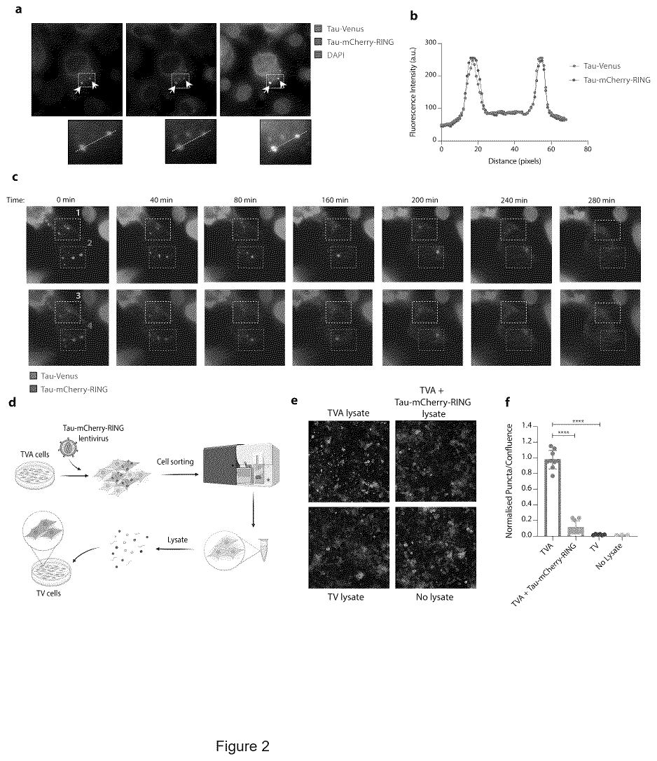

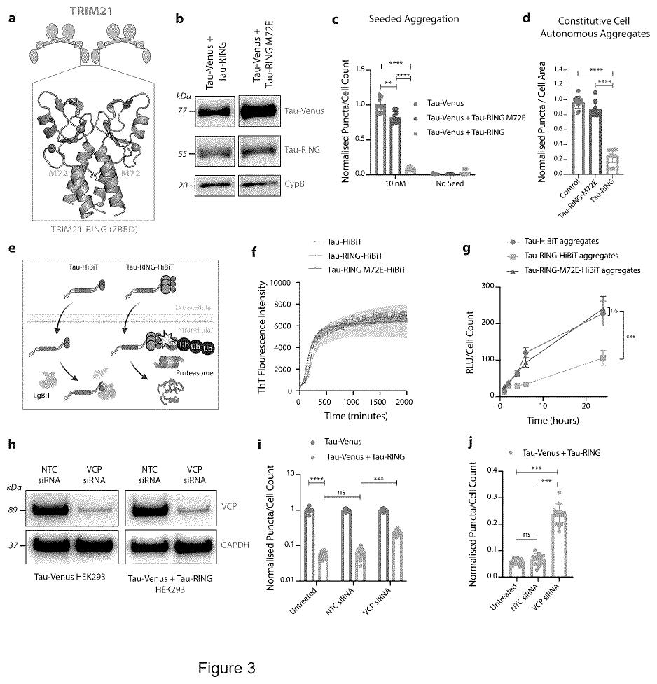

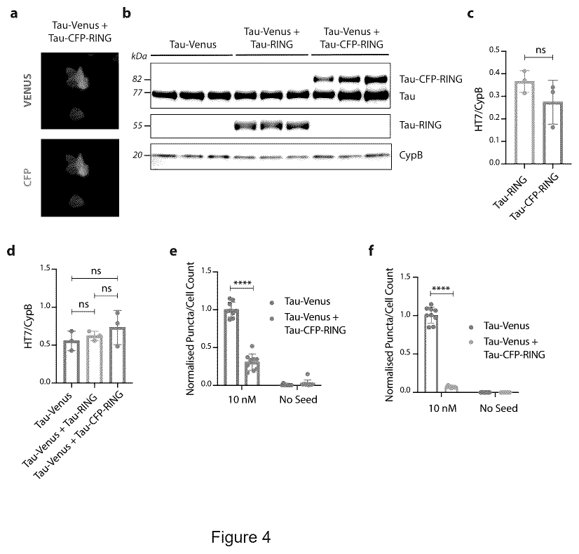

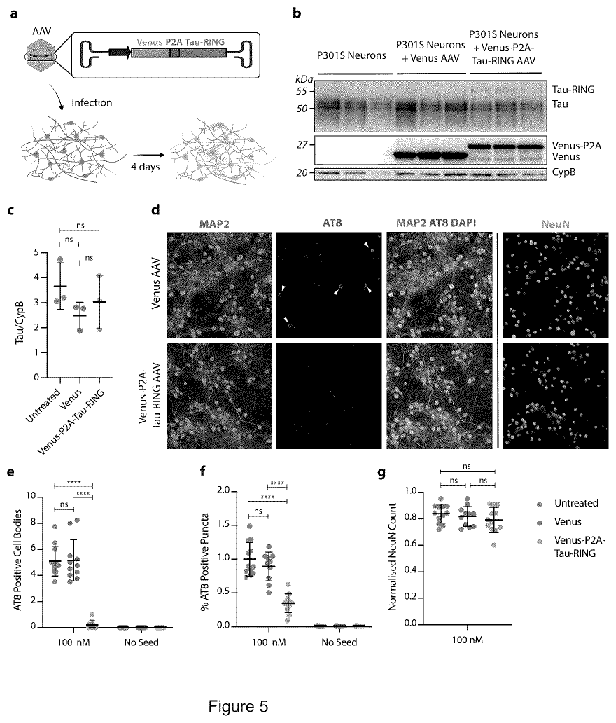

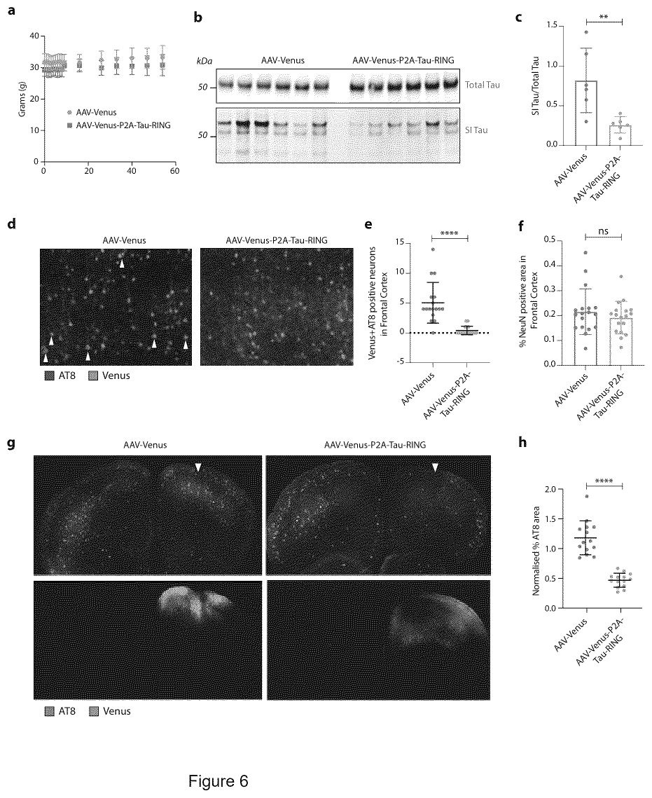

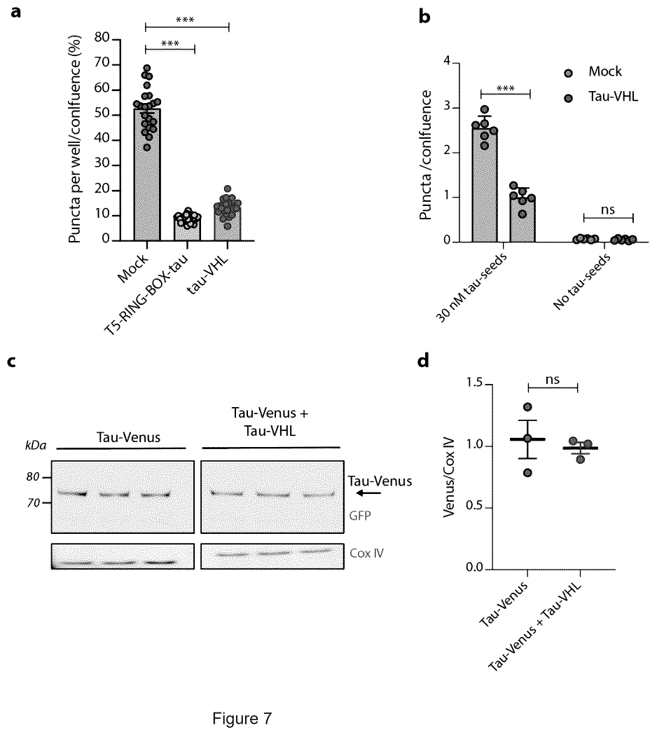

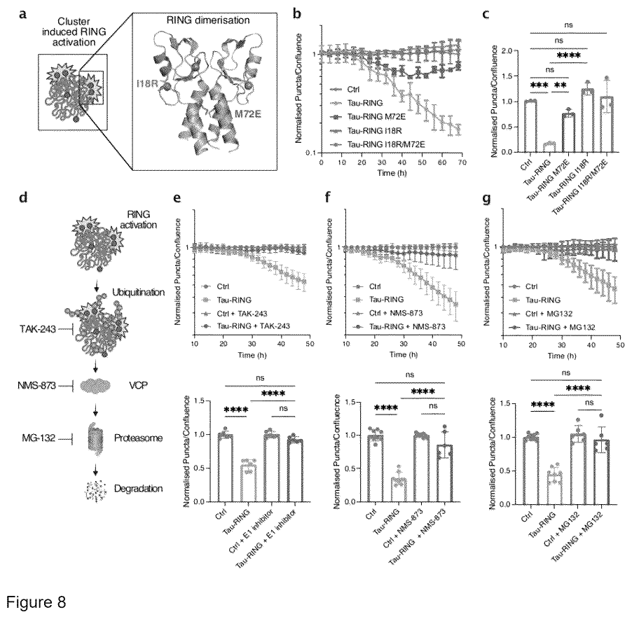

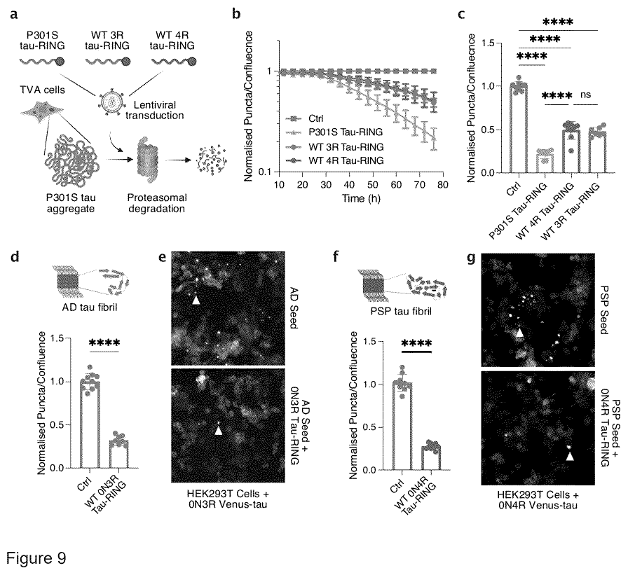

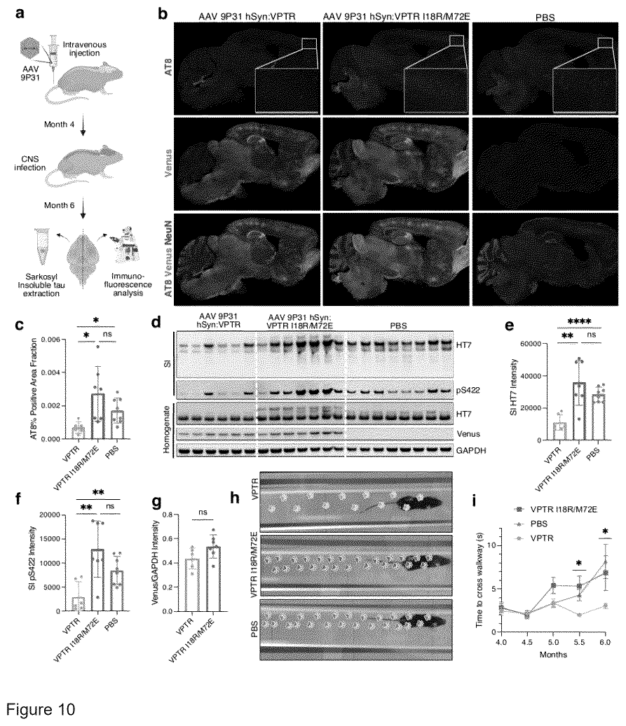

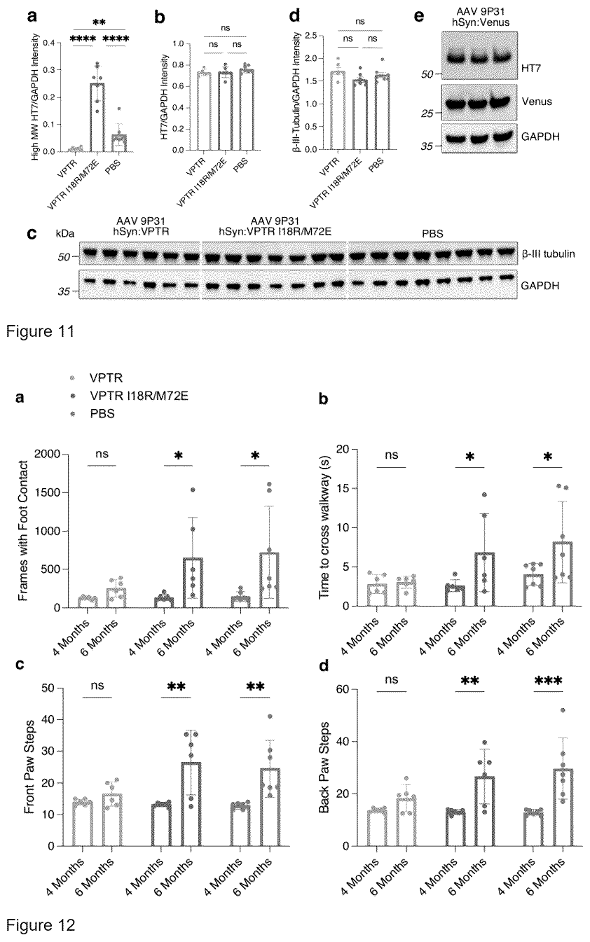

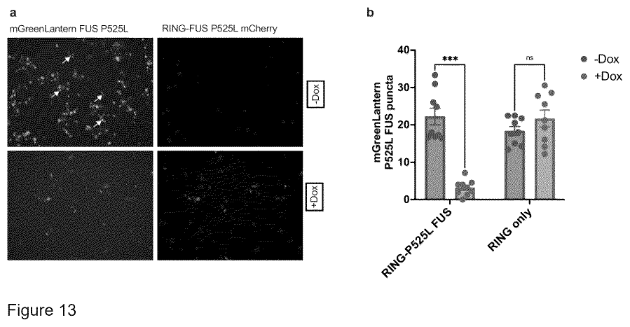

The above data are consistent with tau-RING directly interacting with tau-venus aggregates leading to their degradation. To test this, we engineered baby hamster kidney (BHK) cells to express tau-venus and a doxycycline-inducible fluorescent version of tau-RING, tau-mCherry-RING. This cell line was chosen owing to its large cytoplasm, allowing real-time monitoring of the fate of cytosolic aggregates. We seeded these cells with pre-formed P301S tau seeds and allowed tau-venus aggregates to establish for 24 hrs prior to the addition of doxycycline. Tau-mCherry-RING was recruited to the site of the existing tau-venus aggregates, consistent with incorporation of both constructs into growing aggregates (FIG. 2a,b). Upon incorporation of tau-mCherry-RING, the aggregates were subsequently resolved over a period of ~5 hrs, with tau-venus and tau-mCherry-RING displaying similar kinetics of degradation (FIG. 2c). These results demonstrate that E3 ligase-bait constructs interact with their targets before the wholesale destruction of the complex.

To determine if the degradation of microscopically detectable tau-venus aggregates resulted in the complete destruction of the aggregate, and did not produce smaller seed competent species which could go on to further propagate, a secondary seeding assay was performed. In this assay the lysate from TVA cells, in the presence or absence of tau-RING, was probed for seed competent species by adding the lysate onto TV cells. Evidence of aggregates was evaluated after 72 hours. TVA cells were infected with tau-mCherry-RING lentivirus and then sorted for red and green cells to generate a pure population (FIG. 2d). Cells expressing both tau-venus and tau-mCherry-RING were expanded and then lysed. TVA+tau-mCherry-RING lysate was compared to TVA and TV only lysate. TVA lysate seeded abundant seeded aggregation in fresh TV cells (FIG. 2e,f). TVA+tau-mCherry-RING lysate seeded~90% less seeded aggregation, demonstrating the tau-venus aggregates which could be observed visually closely correlate with the number of seed competent species present within cells (FIG. 2e,f). TV lysate seeded no aggregation, demonstrating tau-venus does not aggregate on its own (FIG. 2e,f). This experiment confirms that tau-RING is initiating the complete degradation of tau aggregates in the cell, and does not cause the formation of small seed competent species.

### Example 2-Proposed Mechanism of Action

Clustering of TRIM21 following ligation of polyvalent immune complexes stimulates E3 catalytic activity by dimerization of the TRIM21 RING domain 12. To determine whether this clustering activity is required for the activity of tau-RING, we repeated our experiments using tau-RING bearing the M72E mutation (tau-RING-M72E) which has been shown to disrupt the RING dimerization interface¹² (FIG. 3a). We stably expressed either tau-RING-M72E or the wildtype equivalent (tau-RING-WT) in TV reporter cells as above (FIG. 3b). Unlike tau-RING-WT, the tau-RING-M72E variant was unable to effectively prevent seeded aggregation or clear existing tau aggregates (FIG. 3c,d). Taken together, the above data support a model wherein tau-RING becomes incorporated into tau aggregates, stimulating RING multimerization and subsequent ubiquitination and degradation. A prediction of this model is that assemblies composed of tau-RING would be short-lived, but that tau assemblies without a RING, or tau-RING-M72E, would remain intact in the cytosol. Recently, we showed that the entry of exogenously supplied tau assemblies to the cytosol can be quantified by luciferase complementation between cytosolically expressed LgBiT and tau bearing the eleven amino acid, HiBiT tag 11 (FIG. 3e). We therefore treated primary mouse neurons with heparin-assembled HiBiT-tagged versions of tau and tau-RING to measure their persistence in the cytosol. Tau-HiBiT, tau-RING-HiBIT and Tau-RING-M72E-HiBiT protein was aggregated in the presence of heparin. All three proteins demonstrated equivalent aggregation dynamics, quantified by Thioflavin T flourescence (FIG. 3f). While tau-HiBiT readily accumulated in the cytosol of primary neurons, substantially lower levels of tau-RING-HiBiT were observed (FIG. 3g). To verify that this difference was not due to failure of tau-RING-HiBIT to be taken up, we also used tau-RING-M72E assemblies. This protein accumulated in a similar manner to tau-HiBiT, confirming the requirement of an intact dimerization interface for efficient degradation of assemblies (FIG. 3g).

TRIM21 can initiate a potent degradation response against virus particles that enter the cytosol with antibodies bound to their capsids, termed antibody-dependent intracellular neutralisation (ADIN). This pathway relies on the ubiquitin-selective AAA+ ATPase and unfoldase, p97/valosin containing protein (VCP), which acts upstream of the proteasome. Inhibition of VCP prevents efficient neutralisation of adenovirus infection by TRIM21 and reduced neutralisation of tau seeding activity in cell based assays (McEwan, William A et al. “Cytosolic Fc receptor TRIM21 inhibits seeded tau aggregation.” Proceedings of the National Academy of Sciences of the United States of America vol. 114, 3 (2017): 574-579. doi: 10.1073/pnas. 1607215114; Hauler, Felix et al. “AAA ATPase p97/VCP is essential for TRIM21-mediated virus neutralization.” Proceedings of the National Academy of Sciences of the United States of America vol. 109, 48 (2012): 19733-8. doi: 10.1073/pnas. 1210659109). We asked whether the same VCP dependency observed for adenovirus neutralisation was also seen in the context of tau-RING degradation. TV cells with and without tau-RING were treated with siRNA to deplete VCP or a non-target control (NTC) (FIG. 3h). Levels of seeded aggregation of tau-venus were subsequently measured in response to exogenously supplied tau assemblies. We found that VCP depletion significantly reduced tau-RING protection relative to control conditions (FIG. 3i). A three-fold increase in seeded aggregation was observed when VCP was depleted (FIG. 3j). These results demonstrate that ADIN and tau-RING share VCP as an essential component for efficient substrate degradation. This data also suggests that tau-RING engages the same degradation machinery as full length TRIM21.

### Example 3: Tau-RING is Effective when Interspaced with a Linker Domain

To investigate if linker size was important for the function of tau-RING, we created a new construct tau-CFP-RING to directly compare to tau-RING. Tau-CFP-RING remained in a soluble state inside cells (FIG. 4a) and was expressed at a similar level to Tau-RING (FIG. 4b,c). No cell autonomous aggregation was observed due to tau over-expression. Similarly to Tau-RING, Tau-CFP-RING did not reduce the level of endogenous tau-venus (FIG. 4d). Tau-CFP-RING was effective at reducing seeded aggregation in TV cells, albeit at lower efficiency (FIG. 4e,f). Domains between the RING and the bait may therefore be incorporated as linkers and retain activity of the construct.

### Example 4—E3 Ligase-Bait Constructs are Effective in Neurons

To validate the efficacy of E3 ligase-bait in a neuronal setting, we produced AAV particles encoding Tau-RING. Due to the reduced efficiency of Tau-RING upon fusion to fluorescent proteins, it was decided to add a P2A cleavage site between the fluorophore and Tau-RING to enable both easy visualisation of construct delivery whilst preserving Tau-RING activity (FIG. 5a). Primary P301S neurons were infected at day 2 with 1×10¹² vgs. The expression of Tau-RING and venus protein was verified by western blot. As with the HEK293 cells, the expression of Tau-RING in neurons did not reduce levels of native soluble tau (FIG. 5b,c). P301S tau aggregates pre-assembled with heparin were added at day 7, and aggregation was analysed at day 14 by AT8 staining (FIG. 5d). Expression of Tau-RING resulted in almost complete prevention of AT8 positive aggregates in cell bodies (FIG. 5e). A substantial reduction of aggregates in neuronal processes was also observed, where approximately 66% reduction was achieved (FIG. 5f). Primary cultures were stained for neuron specific antigen NeuN to determine if there was any cell death caused by either the delivery of the construct, or it's activation upon the addition of tau aggregates. We observed a reduction in cell count in all conditions upon the addition of tau aggregates, however there was no independent cell death caused by the delivery of the viral constructs or the degradation process initiated by Tau-RING (FIG. 5g). These results demonstrate that Tau-RING can be used to prevent the seeded aggregation of tau in primary neurons.

### Example 5—E3 Ligase-Bait is Effective In Vivo

Having determined that E3 ligase-bait is effective at reducing seeded aggregation in cultured neurons, we delivered E3 ligase-bait to an animal disease model of tauopathy. P301S mice develop aggregation predominantly in the spinal cord, brain stem and frontal cortex. Pathology in these areas is developed at 6 months and mice begin to develop motor symptoms, therefore this timepoint was chosen as the endpoint for all experiments. Venus or venus-P2A-Tau-RING was packaged in an AAV vector 9P31, which has recently been demonstrated to cross the blood brain barrier independently. Mice were injected in the tail vein at 4 months with 1×10¹¹ vgs, and culled at 6 months to evaluate tau pathology. No adverse side effects were observed upon delivery of the virus or the constructs they contained, as demonstrated by the steady weight of all mice over the two month experiment (FIG. 6a). Sarkosyl insoluble (SI) tau was extracted from one hemisphere of each brain, and visualised by western blot (FIG. 6b). A significant decrease in SI tau was observed in mice injected with venus-P2A-tau-RING compared with venus only (FIG. 6c). Since complete infection of all neurons cannot be achieved, immunofluorescence staining for tau aggregates by AT8 in the brains was carried out to understand the relationship between infection and the location of tau aggregates (FIG. 6d). Neurons positive for both AT8 and venus were counted in the frontal cortex. Co-localisation was observed in the frontal cortex of all mice infected with venus AAV, however almost no co-localisation occurred between AT8 and venus-P2A-tau-RING (FIG. 6e). This indicates where the venus-P2A-tau-RING virus has infected neurons, aggregates either do not arise or are removed. In the same region of interest evaluated for AT8+venus positive cells, neuronal nuclei were counted. The same denisity of neurons was present in both the control venus group and venus-P2A-tau-RING infected mice, demonstrating the degradation induced by tau-RING does not cause neuronal cell death in vivo (FIG. 6f).

The natural variation in tau pathology between P301S mice is significant. We therefore stereotaxically injected tau-RING into one hemisphere of 5 month old P301S mice, enabling the quantification of the number of tau aggregates to be controlled within each mouse. To see if Tau-RING could remove existing aggregates in vivo, 5-month-old mice were injected in the frontal cortex with 5×10¹⁰ vgs of Venus-P2A-Tau-RING or Venus only AAV PHP.eB and culled at 6 months (FIG. 6g). Injected hemispheres were compared to the contralateral uninjected hemisphere. Analysis of AT8 positive aggregates showed a roughly 50% reduction in AT8 positive area upon delivery of Venus-P2A-Tau-RING (FIG. 6h). Comparison of individual injected versus contralateral hemispheres showed a consistent decrease in AT8 area in all Tau-RING treated mice, in comparison to Venus only treated mice (FIG. 6h). Tau-RING is therefore able to protect against tau pathology in vivo.

### Example 6: Tau Fusions to Other RING Constructs or to E3 Recruiting Scaffolds Permits Removal of Existing Intracellular Tau Aggregates

We next investigated whether the fusion of tau with another RING domain was capable of removing pre-existing tau aggregates. In order to do this, we took advantage of the RING domain of TRIM5×. TRIM5× can form a clustered cage-like structure enclosing retroviral cores in the cytoplasm, leading to the activation of its RING E3 ligase domains, triggering the degradation of the viral particle. The Box domain of TRIM5× is known to assist in the clustering of TRIM5×. We therefore fused tau with the RING-Box domains of TRIM5×. Lentivirus containing P301S T5-RING-Box-Tau was applied to TVA cells. T5-RING-Box-Tau reduced pre-existing aggregates by ~80% (FIG. 7a), further suggesting that other clustering-RINGs may be exploited to achieve tau aggregate removal.

The data showed so far demonstrated that direct fusion of a RING to a bait is able to promote degradation of assembled targets.

We next tested whether fusion of bait to a domain which recruits an E3 would also suffice. To do so, we fused tau to the von Hippel-Lindau tumour suppressor protein (VHL), which recruits the cullin-E3 ligase complex. TVA cells infected with a lentivirus coding for tau-VHL showed a ~5 times reduction in tau aggregates (FIG. 7a). Furthermore, when recombinant 0N4R P301S tau seeds were transfected into TV cells, an inhibition of the seeded aggregation of tau occurred when Tau-VHL was expressed (FIG. 7b) without any change in the expression of monomeric tau (FIG. 7c,d). Thus, both direct fusion to an E3 and fusion to an E3-recruiting protein can induce degradation of assembled targets without compromising the stability of their monomeric form.

### Example 7—Discussion of Examples 1 to 6

We have here demonstrated that a fusion between an E3 catalytic domain or a recruiting protein or domain and a protein with the capacity to form pathological aggregates can enable the selective degradation of assembled proteins. This was exemplified here with tau, which can adopt filamentous conformations during neurodegenerative disease, a property shared with other proteins implicated in neurodegeneration such as TDP43 and α-synuclein.

We showed that incorporation of Tau-RING into tau filaments promotes degradation of the filaments, leading to potent protection against seeded. The Tau-RING constructs were also capable of degrading existing aggregates within cells. This demonstrates that the ubiquitin-proteasome system is able to process and remove these large aggregates, provided the correct signal is present.

We further showed that this protection was conferred to neurons expressing Tau-RING constructs in cell-based assays of seeded aggregation and following AAV-mediated expression in the mouse brain. Thus neurons, like the HEK293 and BHK cell lines, possess the machinery necessary to promote clearance of filamentous tau structures.

The activation of Tau-RING was dependent on clustering of RING domains following incorporation of Tau-RING into filaments. Overall, our results demonstrate that effective protection against protein misfolding and aggregation can be achieved using the E3 ligase-bait strategy.

### Example 8—the Mechanism of RING-Bait is Driven by RING Activity

FIG. 8 illustrates how the RING-Bait technology is driven by RING activity by use of functional mutations and inhibitors. The data demonstrates that the process requires RING to both dimerise (M72E) and bind ubiquitin (118R) (see FIG. 8A-C). Furthermore the RING-Bait mechanism proceeds via ubiquitination (which is blocked by E1 inhibitor TAK-243), the unfoldase/segragase VCP (blocked by NMS-873) and then the proteasome (blocked by MG132) (FIG. 8D-G).

### Example 9—RING-Bait can be Utilised with Different Portions of the Bait Protein and can Target Different Types of Aggregate

FIG. 9 illustrates how different isoforms of tau (3R and 4R) can both be used as bait fragments-see FIG. 9A-C. Furthermore RING-bait is effective against the different types of tau aggregates induced by Alzheimer's disease (AD) or progressive supranuclear palsy (PSP) (FIGS. 9D-E and 9F-G respectively).

### Example 10—RING-Bait can be Used In Vivo to Improve Clinical Phenotype

FIG. 10 shows in vivo data demonstrating effectiveness of RING-bait not just against tau aggregates in mouse brains, but also in achieving positive behavioural effects. Active RING-Bait or catalytically-inactive RING-Bait (118R/M72E) was delivered by AAV at 4 months to P301S mice, then brains were analysed by immunofluorescence (FIG. 10B&C) or western blot (FIG. 10D-G; FIG. 11). Active RING-Bait reduced tau aggregates by both measures. Furthermore, behavioural data shows that mice treated with RING-Bait had an improved motor phenotype at 6 months compared to untreated control or inactive control (FIG. 10H&I; FIG. 12).

### Example 11—Different Types of Protein can be Used as Bait in RING-Bait

FIG. 13 confirms that a mutant form of Fused in sarcoma (FUS) protein that aggregates (P525L) can be degraded using a RING-bait construct (RING-FUS P525L). The RING-FUS construct was expressed with a self-cleaving T2A peptide and mCherry at the C-terminus (see SEQ ID NO: 7).

### REFERENCES

1. Shi, Y. et al. Structure-based classification of tauopathies. Nature 598, 359-363 (2021).
2. Arseni, D. et al. Structure of pathological TDP-43 filaments from ALS with FTLD. Nature 1-5 (2021) doi: 10.1038/s41586-021-04199-3.
3. Schweighauser, M. et al. Structures of α-synuclein filaments from multiple system atrophy. Nature 585, 464-469 (2020).
4. Biogen Shelves Gosuranemab After Negative Alzheimer's Trial|ALZFORUM. https://www.alzforum.org/news/research-news/biogen-shelves-gosuranemab-after-negative-alzheimers-trial.
5. Meisl, G. et al. In vivo rate-determining steps of tau seed accumulation in Alzheimer's disease. Science Advances (2021) doi: 10.1126/sciadv.abh1448.
6. Hyun, S. & Shin, D. Chemical-Mediated Targeted Protein Degradation in Neurodegenerative Diseases. Life 11, 607 (2021).
7. Zeng, J. et al. Target-induced clustering activates Trim-Away of pathogens and proteins. Nat Struct Mol Biol 28, 278-289 (2021).
8. McEwan, W. A. et al. Cytosolic Fc receptor TRIM21 inhibits seeded tau aggregation. Proc Natl Acad Sci USA 114, 574-579 (2017).
9. Croft, C. L. et al. Photodynamic studies reveal rapid formation and appreciable turnover of tau inclusions. Acta Neuropathol 141, 359-381 (2021).
10. Kundel, F. et al. Measurement of Tau Filament Fragmentation Provides Insights into Prion-like Spreading. ACS Chem Neurosci 9, 1276-1282 (2018).
11. Tuck, B. J. et al. Tau assemblies enter the cytosol in a cholesterol sensitive process essential to seeded aggregation. 2021.06.21.449238 https://www.biorxiv.org/content/10.1101/2021.06.21.449238v3 (2021) doi: 10.1101/2021.06.21.449238.
12. Dickson, C. et al. Intracellular antibody signalling is regulated by phosphorylation 12. of the Fc receptor TRIM21. eLife 7, e32660.
13. Zhang, W. et al. Heparin-induced tau filaments are polymorphic and differ from those in Alzheimer's and Pick's diseases. eLife 8, e43584 (2019).
14. Goodwin, M. S. et al. Anti-tau scFvs Targeted to the Cytoplasm or Secretory Pathway Variably Modify Pathology and Neurodegenerative Phenotypes. Molecular Therapy 29, 859-872 (2021).
15. Danis, C. et al. Inhibition of Tau seeding by targeting Tau nucleation core within neurons with a single domain antibody fragment. 2021.03.23.436266 https://www.biorxiv.org/content/10.1101/2021.03.23.436266v1 (2021) doi: 10.1101/2021.03.23.436266.
16. Gallardo, G. et al. Targeting tauopathy with engineered tau-degrading intrabodies. Molecular Neurodegeneration 14, 38 (2019).
17. Lu, M. et al. Discovery of a Keap1-dependent peptide PROTAC to knockdown Tau by ubiquitination-proteasome degradation pathway. Eur J Med Chem 146, 251-259 (2018).
18. Chu, T.-T. et al. Specific Knockdown of Endogenous Tau Protein by Peptide-Directed Ubiquitin-Proteasome Degradation. Cell Chemical Biology 23, 453-461 (2016).
19. Silva, M. C. et al. Targeted degradation of aberrant tau in frontotemporal dementia patient-derived neuronal cell models. eLife 8, e45457 (2019).

TABLE 1

| | Gene | Example | |
|---|---|---|---|
| | ID | transcript | Protein sequence containing RING domain |
| RNF4 | 6047 | NM_ | SGTVSCPICMDGYSEIVQNGRLIVSTECGHVFCSQCLRDSLKNANTCPTCRKKINHK |
| | | 001185009.3 | |
| XIAP | 331 | NM_001167.4 | EISTEEQLRRLQEEKLCKICMDRNIAIVFVPCGHLVTCKQCAEAVDKCPMCYTVITFKQKIF |
| | | | MS |
| TRIM5 | 85363 | NM_033034.3 | EVTCPICLELLTQPLSLDCGHSFCQACLTANHKKSMLDKGESSCPVCRI |
| TRIM7 | 81786 | NM_203293.3 | QGEATCSICLELFREPVSVECGHSFCRACIGRCWERPGAGSVGAATRAPPFPLPCPQCREPAR |
| | | | PSQLRPNR |
| MDM2 | 4193 | NM_002392.6 | IEPCVICQGRPKNGCIVHGKTGHLMACFTCAKKLKKRNKPCPVCRQPIQMIVLTYFP |
| CBL | 867 | NM_005188.4 | QDHIKVTQEQYELYCEMGSTFQLCKICAENDKDVKIEPCGHLMCTSCLTSWQESEGQGCPFC |
| | | | RCEIKGTEPIVVDPF |
| BRCA1 | 672 | NM_007294.4 | RVEEVQNVINAMQKILECPICLELIKEPVSTKCDHIFCKFCMLKLLNQKKGPSQCPLCKNDI |
| | | | TKRSLQESTRESQLVEELLKIICAFQLDTGL |
| STUB1 | 10273 | NM_005861.4 | IPDYLCGKISFELMREPCITPSGITYDRKDIEEHLQRVGHFDPVTRSPLTQEQLIPNLAMKE |
| (CHIP) | | | VIDAFISEN |
| RBX1 | 9978 | NM_014248.4 | DNCAICRNHIMDLCIECQANQASATSEECTVAWGVCNHAFHFHCISRWLKTRQVCPLDNREW |
| TRIM21 | 6737 | NM_003141.4 | LTMMWEEVTCPICLDPFVEPVSIECGHSFCQECISQVGKGGGSVCPVCRQRFLLKNLRPNRQ |
| | | | LANMVNNLKEISQ |
| BARD1 | 580 | NM_000465.4 | AWAHSRAALDRLEKLLRCSRCTNILREPVCLGGCEHIFCSNCVSDCIGTGCPVCYTPAWIQD |
| | | | LKINRQLDSMIQLCSKLRNLLHDN |
| HTLF | 6596 | NM_ | GSDEECAICLDSLTVPVITHCAHVFCKPCICQVIQNEQPHAKCPLCRNDIHEDNL |
| | | 001318935.2 | |
| NFX1 | 4799 | NM_002504.6 | YECMVCCELVRVTAPVWSCQSCYHVFHLNCIKKWARSPASQADGQSGWRCPACQ |
| TRIM56 | 81844 | NM_030961.3 | FLACKICLEQLRAPKTLPCLHTYCQDCLAQLADGGRVRCPECRETVPVPPEGVASF |

TABLE 3

| Gene | Gene | Gene | | |
|---|---|---|---|---|
| name | ID | accession | mRNA accession | Example CDS Sequence |
| MAPT | 4137 | NG_ | 4R tau isoforms: | ″MAEPRQEFEVMEDHAGTYGLGDRKDQGGYTMHQDQEGDTDAGLK |
| | | 007398.2 | NM_005910.6, | ESPLQTPTEDGSEEPGSETSDAKSTPTAEDVTAPLVDEGAPGKQAAAQPHTEIPEGTT |
| | | | NM_001123067.4, | AEEAGIGDTPSLEDEAAGHVTQEPESGKVVQEGFLREPGPPGLSHQLMSGMPGAPLLP |
| | | | NM_016834.5 | EGPREATRQPSGTGPEDTEGGRHAPELLKHQLLGDLHQEGPPLKGAGGKERPGSKEEV |
| | | | | DEDRDVDESSPQDSPPSKASPAQDGRPPQTAAREATSIPGFPAEGAIPLPVDFLSKVS |
| | | | | TEIPASEPDGPSVGRAKGQDAPLEFTFHVEITPNVQKEQAHSEEHLGRAAFPGAPGEG |
| | | | | PEARGPSLGEDTKEADLPEPSEKQPAAAPRGKPVSRVPQLKARMVSKSKDGTGSDDKK |
| | | | | AKTSTRSSAKTLKNRPCLSPKHPTPGSSDPLIQPSSPAVCPEPPSSPKYVSSVTSRTG |
| | | | | SSGAKEMKLKGADGKTKIATPRGAAPPGQKGQANATRIPAKTPPAPKTPPSSGEPPKS |
| | | | | GDRSGYSSPGSPGTPGSRSRTPSLPTPPTREPKKVAVVRTPPKSPSSAKSRLQTAPVP |
| | | | | MPDLKNVKSKIGSTENLKHQPGGGKVQIINKKLDLSNVQSKCGSKDNIKHVPGGGSVQ |
| | | | | IVYKPVDLSKVTSKCGSLGNIHHKPGGGQVEVKSEKLDFKDRVQSKIGSLDNITHVPG |
| | | | | GGNKKIETHKLTFRENAKAKTDHGAEIVYKSPVVSGDTSPRHLSNVSSTGSIDMVDSP |
| | | | | QLATLADEVSASLAKQGL″ |
| MAPT | 4137 | NG_ | 3R tau isoforms: | |
| | | 007398.2 | NM_001203252.2, | |
| | | | NM_001203251.2, | |
| | | | NM_016841.5 | |
| MAPT | 4137 | NG_ | 3R and 4R tau | |
| | | 007398.2 | isoforms: | |
| | | | NM_005910.6, | |
| | | | NM_001123067.4, | |
| | | | NM_016834.5, | |
| | | | NM_001203252.2, | |
| | | | NM_001203251.2, | |
| | | | NM_016841.5 | |
| TARDBP | 23435 | NG_ | NM_007375.4 | ″MSEYIRVTEDENDEPIEIPSEDDGTVLLSTVTAQFPGACGLRYR |
| | | 008734.1 | | NPVSQCMRGVRLVEGILHAPDAGWGNLVYVVNYPKDNKRKMDETDASSAVKVKRAVQK |
| | | | | TSDLIVLGLPWKTTEQDLKEYFSTFGEVLMVQVKKDLKTGHSKGFGFVRFTEYETQVK |
| | | | | VMSQRHMIDGRWCDCKLPNSKQSQDEPLRSRKVFVGRCTEDMTEDELREFFSQYGDVM |
| | | | | DVFIPKPFRAFAFVTFADDQIAQSLCGEDLIIKGISVHISNAEPKHNSNRQLERSGRE |
| | | | | GGNPGGFGNQGGFGNSRGGGAGLGNNQGSNMGGGMNEGAFSINPAMMAAAQAALQSSW |
| | | | | GMMGMLASQQNQSGPSGNNQNQGNMQREPNQAFGSGNNSYSGSNSGAAIGWGSASNAG |
| | | | | SGSGENGGFGSSMDSKSSGWGM″ |
| SNCA | 6622 | NG_ | NM_000345.4, | ″MDVFMKGLSKAKEGVVAAAEKTKQGVAEAAGKTKEGVLYVGSKT |
| | | 011851.1 | NM_007308.3, | KEGVVHGVATVAEKTKEQVTNVGGAVVTGVTAVAQKTVEGAGSIAAATGFVKKDQLGK |
| | | | NM_001375290.1 | NEEGAPQEGILEDMPVDPDNEAYEMPSEEGYQDYEPEA″ |
| HTT | 3064 | NG_ | NM_002111.8, | ″MATLEKLMKAFESLKSFQQQQQQQQQQQQQQQPPPP |
| | | 009378.1 | NM_001388492.1 | PPPPPPPQLPQPPPQAQPLLPQPQPPPPPPPPPPGPAVAEEPLHRPKKELSATKKDRV |
| | | | | NHCLTICENIVAQSVRNSPEFQKLLGIAMELFLLCSDDAESDVRMVADECLNKVIKAL |
| | | | | MDSNLPRLQLELYKEIKKNGAPRSLRAALWRFAELAHLVRPQKCRPYLVNLLPCLTRT |
| | | | | SKRPEESVQETLAAAVPKIMASFGNFANDNEIKVLLKAFIANLKSSSPTIRRTAAGSA |
| | | | | VSICQHSRRTQYFYSWLLNVLLGLLVPVEDEHSTLLILGVLLTLRYLVPLLQQQVKDT |
| | | | | SLKGSFGVTRKEMEVSPSAEQLVQVYELTLHHTQHQDHNVVTGALELLQQLFRTPPPE |
| | | | | LLQTLTAVGGIGQLTAAKEESGGRSRSGSIVELIAGGGSSCSPVLSRKQKGKVLLGEE |
| | | | | EALEDDSESRSDVSSSALTASVKDEISGELAASSGVSTPGSAGHDIITEQPRSQHTLQ |
| | | | | ADSVDLASCDLTSSATDGDEEDILSHSSSQVSAVPSDPAMDLNDGTQASSPISDSSQT |
| | | | | TTEGPDSAVTPSDSSEIVLDGTDNQYLGLQIGQPQDEDEEATGILPDEASEAFRNSSM |
| | | | | ALQQAHLLKNMSHCRQPSDSSVDKFVLRDEATEPGDQENKPCRIKGDIGQSTDDDSAP |
| | | | | LVHCVRLLSASFLLTGGKNVLVPDRDVRVSVKALALSCVGAAVALHPESFFSKLYKVP |
| | | | | LDTTEYPEEQYVSDILNYIDHGDPQVRGATAILCGTLICSILSRSRFHVGDWMGTIRT |
| | | | | LTGNTFSLADCIPLLRKTLKDESSVTCKLACTAVRNCVMSLCSSSYSELGLQLIIDVL |
| | | | | TLRNSSYWLVRTELLETLAEIDERLVSFLEAKAENLHRGAHHYTGLLKLQERVLNNVV |
| | | | | IHLLGDEDPRVRHVAAASLIRLVPKLFYKCDQGQADPVVAVARDQSSVYLKLLMHETQ |
| | | | | PPSHFSVSTITRIYRGYNLLPSITDVTMENNLSRVIAAVSHELITSTTRALTFGCCEA |
| | | | | LCLLSTAFPVCIWSLGWHCGVPPLSASDESRKSCTVGMATMILTLLSSAWFPLDLSAH |
| | | | | QDALILAGNLLAASAPKSLRSSWASEEEANPAATKQEEVWPALGDRALVPMVEQLFSH |
| | | | | LLKVINICAHVLDDVAPGPAIKAALPSLTNPPSLSPIRRKGKEKEPGEQASVPLSPKK |
| | | | | GSEASAASRQSDTSGPVTTSKSSSLGSFYHLPSYLKLHDVLKATHANYKVTLDLQNST |
| | | | | EKFGGFLRSALDVLSQILELATLQDIGKCVEEILGYLKSCFSREPMMATVCVQQLLKT |
| | | | | LEGTNLASQFDGLSSNPSKSQGRAQRLGSSSVRPGLYHYCFMAPYTHETQALADASLR |
| | | | | NMVQAEQENDTSGWFDVLQKVSTQLKTNLTSVTKNRADKNAIHNHIRLFEPLVIKALK |
| | | | | QYTTTTCVQLQKQVLDLLAQLVQLRVNYCLLDSDQVFIGFVLKQFEYIEVGQFRESEA |
| | | | | IIPNIFFFLVLLSYERYHSKQIIGIPKIIQLCDGIMASGRKAVTHAIPALQPIVHDLE |
| | | | | VLRGTNKADAGKELETQKEVVVSMLLRLIQYHQVLEMFILVLQQCHKENEDKWKRLSR |
| | | | | QIADIILPMLAKQQMHIDSHEALGVLNTLFEILAPSSLRPVDMLLRSMFVTPNTMASV |
| | | | | STVQLWISGILAILRVLISQSTEDIVLSRIQELSFSPYLISCTVINRLRDGDSTSTLE |
| | | | | EHSEGKQIKNLPEETFSRELLQLVGILLEDIVTKQLKVEMSEQQHTFYCQELGTLLMC |
| | | | | LIHIFKSGMERRITAAATRLFRSDGCGGSFYTLDSLNLRARSMITTHPALVLLWCQIL |
| | | | | LLVNHTDYRWWAEVQQTPKRHSLSSTKLLSPQMSGEEEDSDLAAKLGMCNREIVRRGA |
| | | | | LILFCDYVCQNLHDSEHLTWLIVNHIQDLISLSHEPPVQDFISAVHRNSAASGLFIQA |
| | | | | IQSRCENLSTPTMLKKTLQCLEGIHLSQSGAVLTLYVDRLLCTPERVLARMVDILACR |
| | | | | RVEMLLAANLQSSMAQLPMEELNRIQEYLQSSGLAQRHQRLYSLLDRERLSTMQDSLS |
| | | | | PSPPVSSHPLDGDGHVSLETVSPDKDWYVHLVKSQCWTRSDSALLEGAELVNRIPAED |
| | | | | MNAFMMNSEFNLSLLAPCLSLGMSEISGGQKSALFEAAREVTLARVSGTVQQLPAVHH |
| | | | | VFQPELPAEPAAYWSKLNDLFGDAALYQSLPTLARALAQYLVVVSKLPSHLHLPPEKE |
| | | | | KDIVKFVVATLEALSWHLIHEQIPLSLDLQAGLDCCCLALQLPGLWSVVSSTEFVTHA |
| | | | | CSLIYCVHFILEAVAVQPGEQLLSPERRTNTPKAISEEEEEVDPNTQNPKYITAACEM |
| | | | | VAEMVESLQSVLALGHKRNSGVPAFLTPLLRNIIISLARLPLVNSYTRVPPLVWKLGW |
| | | | | SPKPGGDFGTAFPEIPVEFLQEKEVFKEFIYRINTLGWTSRTQFEETWATLLGVLVTQ |
| | | | | PLVMEQEESPPEEDTERTQINVLAVQAITSLVLSAMTVPVAGNPAVSCLEQQPRNKPL |
| | | | | KALDTRFGRKLSIIRGIVEQEIQAMVSKRENIATHHLYQAWDPVPSLSPATTGALISH |
| | | | | EKLLLQINPERELGSMSYKLGQVSIHSVWLGNSITPLREEEWDEEEEEEADAPAPSSP |
| | | | | PTSPVNSRKHRAGVDIHSCSQFLLELYSRWILPSSSARRTPAILISEVVRSLLVVSDL |
| | | | | FTERNQFELMYVTLTELRRVHPSEDEILAQYLVPATCKAAAVLGMDKAVAEPVSRLLE |
| | | | | STLRSSHLPSRVGALHGVLYVLECDLLDDTAKQLIPVISDYLLSNLKGIAHCVNIHSQ |
| | | | | QHVLVMCATAFYLIENYPLDVGPEFSASIIQMCGVMLSGSEESTPSIIYHCALRGLER |
| | | | | LLLSEQLSRLDAESLVKLSVDRVNVHSPHRAMAALGLMLTCMYTGKEKVSPGRTSDPN |
| | | | | PAAPDSESVIVAMERVSVLFDRIRKGFPCEARVVARILPQFLDDFFPPQDIMNKVIGE |
| | | | | FLSNQQPYPQFMATVVYKVFQTLHSTGQSSMVRDWVMLSLSNFTQRAPVAMATWSLSC |
| | | | | FFVSASTSPWVAAILPHVISRMGKLEQVDVNLFCLVATDFYRHQIEEELDRRAFQSVL |
| | | | | EVVAAPGSPYHRLLTCLRNVHKVTTC″ |
| RHO | 6010 | NG_ | NM_000539.3 | ″MNGTEGPNFYVPFSNATGVVRSPFEYPQYYLAEPWQFSMLAAYM |
| | | 009115.1 | | FLLIVLGFPINFLTLYVTVQHKKLRTPLNYILLNLAVADLFMVLGGFTSTLYTSLHGY |
| | | | | FVFGPTGCNLEGFFATLGGEIALWSLVVLAIERYVVVCKPMSNFRFGENHAIMGVAFT |
| | | | | WVMALACAAPPLAGWSRYIPEGLQCSCGIDYYTLKPEVNNESFVIYMFVVHFTIPMII |
| | | | | IFFCYGQLVFTVKEAAAQQQESATTQKAEKEVTRMVIIMVIAFLICWVPYASVAFYIF |
| | | | | THQGSNFGPIFMTIPAFFAKSAAIYNPVIYIMMNKQFRNCMLTTICCGKNPLGDDEAS |
| | | | | ATVSKTETSQVAPA″ |
| SOD1 | 6647 | NG_ | NM_000454.5 | ″MATKAVCVLKGDGPVQGIINFEQKESNGPVKVWGSIKGLTEGLH |
| | | 008689.1 | | GFHVHEFGDNTAGCTSAGPHENPLSRKHGGPKDEERHVGDLGNVTADKDGVADVSIED |
| | | | | SVISLSGDHCIIGRTLVVHEKADDLGKGGNEESTKTGNAGSRLACGVIGIAQ″ |
| FUS | 2521 | NG_ | NM_004960.4 | ″MASNDYTQQATQSYGAYPTQPGQGYSQQSSQPYGQQSYSGYSQS |
| | | 012889.2 | NM_001170937.1 | TDTSGYGQSSYSSYGQSQNTGYGTQSTPQGYGSTGGYGSSQSSQSSYGQQSSYPGYGQ |
| | | | NM_001170634.1 | QPAPSSTSGSYGSSSQSSSYGQPQSGSYSQQPSYGGQQQSYGQQQSYNPPQGYGQQNQ |
| | | | | YNSSSGGGGGGGGGGNYGQDQSSMSSGGGSGGGYGNQDQSGGGGSGGYGQQDRGGRGR |
| | | | | GGSGGGGGGGGGGYNRSSGGYEPRGRGGGRGGRGGMGGSDRGGFNKFGGPRDQGSRHD |
| | | | | SEQDNSDNNTIFVQGLGENVTIESVADYFKQIGIIKTNKKTGQPMINLYTDRETGKLK |
| | | | | GEATVSFDDPPSAKAAIDWFDGKEFSGNPIKVSFATRRADFNRGGGNGRGGRGRGGPM |
| | | | | GRGGYGGGGSGGGGRGGFPSGGGGGGGQQRAGDWKCPNPTCENMNFSWRNECNQCKAP |
| | | | | KPDGPGGGPGGSHMGGNYGDDRRGGRGGYDRGGYRGRGGDRGGFRGGRGGGDRGGFGP |
| | | | | GKMDSRGEHRQDRRERPY″ |

Other sequences
TRIM21
(SEQ ID NO: 1)
MASAARLTMMWEEVTCPICLDPFVEPVSIECGHSFCQECISQVGKGGGS
VCPVCRQRFLLKNLRPNRQLANMVNNLKEISQEAREGTQGERCAVHGER
LHLFCEKDGKALCWVCAQSRKHRDHAMVPLEEAAQEYQEKLQVALGELR
RKQELAEKLEVEIAIKRADWKKTVETQKSRIHAEFVQQKNFLVEEEQRQ
LQELEKDEREQLRILGEKEAKLAQQSQALQELISELDRRCHSSALELLQ
EVIIVLERSESWNLKDLDITSPELRSVCHVPGLKKMLRTCAVHITLDPD
TANPWLILSEDRRQVRLGDTQQSIPGNEERFDSYPMVLGAQHFHSGKHY
WEVDVTGKEAWDLGVCRDSVRRKGHFLLSSKSGFWTIWLWNKQKYEAGT
YPQTPLHLQVPPCQVGIFLDYEAGMVSFYNITDHGSLIYSFSECAFTGP
LRPFFSPGFNDGGKNTAPLTLCPLNIGSQGSTDY
Amino acids 1-85 of TRIM21
(SEQ ID NO: 2)
MASAARLTMMWEEVTCPICLDPFVEPVSIECGHSFCQECISQVGKGGGS
VCPVCRQRFLLKNLRPNRQLANMVNNLKEISQEARE
***

Tau-RING amino acid sequence:
(SEQ ID NO: 3)
N′...MAEPRQEFEVMEDHAGTYGLGDRKDQGGYTMHQDQEGDTDAGLKAEEAGIGDTPSLEDEAAGH
VTQARMVSKSKDGTGSDDKKAKGADGKTKIATPRGAAPPGQKGQANATRIPAKTPPAPKTPPSSGE
PPKSGDRSGYSSPGSPGTPGSRSRTPSLPTPPTREPKKVAVVRTPPKSPSSAKSRLQTAPVPMPDL
KNVKSKIGSTENLKHQPGGGKVQIINKKLDLSNVQSKCGSKDNIKHVSGGGSVQIVYKPVDLSKVT
SKCGSLGNIHHKPGGGQVEVKSEKLDFKDRVQSKIGSLDNITHVPGGGNKKIETHKLTFRENAKAK

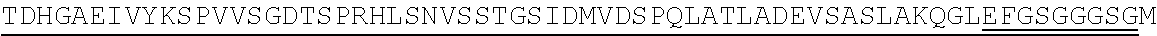

ASAARLTMMWEEVTCPICLDPFVEPVSIECGHSFCQECISQVGKGGGSVCPVCRQRFLLKNLRPNR
QLANMVNNLKEISQEARE...′C
Tau (0N4R P301S)
Linker (EcoR1 site and GSGGGSG flexible region)
RING (TRIM21 RING domain)
***

Tau-CFP-RING amino acid sequence
(SEQ ID NO: 4)
N′...MAEPRQEFEVMEDHAGTYGLGDRKDQGGYTMHQDQEGDTDAGLKAEEAGIGDTPSLEDEAAGH
VTQARMVSKSKDGTGSDDKKAKGADGKTKIATPRGAAPPGQKGQANATRIPAKTPPAPKTPPSSGE
PPKSGDRSGYSSPGSPGTPGSRSRTPSLPTPPTREPKKVAVVRTPPKSPSSAKSRLQTAPVPMPDL
KNVKSKIGSTENLKHQPGGGKVQIINKKLDLSNVQSKCGSKDNIKHVSGGGSVQIVYKPVDLSKVT
SKCGSLGNIHHKPGGGQVEVKSEKLDFKDRVQSKIGSLDNITHVPGGGNKKIETHKLTFRENAKAK

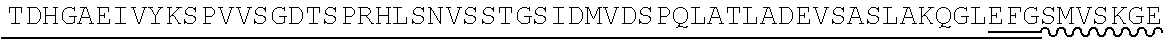

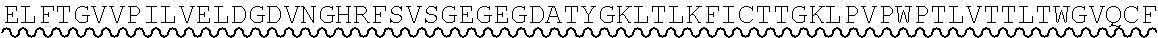

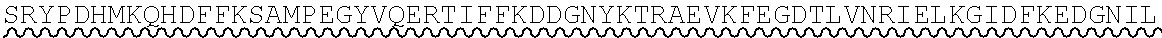

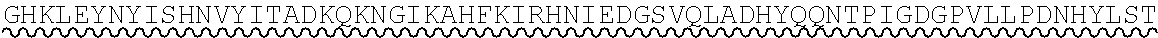

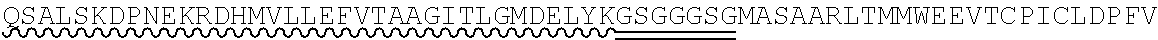

EPVSIECGHSFCQECISQVGKGGGSVCPVCRQRFLLKNLRPNRQLANMVNNLKEISQEARE...′C
Tau (0N4R P301S)
Linker 1 (EcoRI + BamHI site)
CFP
Linker 2 (GSGGGSG flexible region)
RING (TRIM21 RING domain)
***

| VHL (Gene ID 7428) | |
|---|---|
| (SEQ ID NO: 5) | |
| *MPRRAENWDEAEVGAEEAGVEEYGPEEDGGEESGAEESGPEESGPEELGAEEEMEAGRPRPVLRS | |
| VNSREPSQVIFCNRSPRVVLPVWLNFDGEPQPYPTLPPGTGRRIHSYRGHLWLFRDAGTHDGLLVN | |
| QTELFVPSLNVDGQPIFANITLPVYTLKERCLQVVRSLVKPENYRRLDIVRSLYEDLEDHPNVQKD | |
| LERLTQERIAHQRMGD* | |
| *** | |
| Trim5-B-box domain | |
| (SEQ ID NO: 6) | |
| QKVDHCARHGEKLLLFCQEDGKVICWLCERSQEHRGHHTFLTE | |
| *** | |
| RING-FUS construct | |
| (SEQ ID NO: 7) | |
| 001 | MASAARLTMM WEEVTCPICL DPFVEPVSIE CGHSFCQECI SQVGKGGGSV CPVCRQRFLL |
| 061 | KNLRPNRQLA NMVNNLKEIS QEAREGGGGS MASNDYTQQA TQSYGAYPTQ PGQGYSQQSS |
| 121 | QPYGQQSYSG YSQSTDTSGY GQSSYSSYGQ SQNTGYGTQS TPQGYGSTGG YGSSQSSQSS |
| 181 | YGQQSSYPGY GQQPAPSSTS GSYGSSSQSS SYGQPQSGSY SQQPSYGGQQ QSYGQQQSYN |
| 241 | PPQGYGQQNQ YNSSSGGGGG GGGGGNYGQD QSSMSSGGGS GGGYGNQDQS GGGGSGGYGQ |
| 301 | QDRGGRGRGG SGGGGGGGGG GYNRSSGGYE PRGRGGGRGG RGGMGGSDRG GFNKFGGPRD |
| 361 | QGSRHDSEQD NSDNNTIFVQ GLGENVTIES VADYFKQIGI IKTNKKTGQP MINLYTDRET |
| 421 | GKLKGEATVS FDDPPSAKAA IDWEDGKEFS GNPIKVSFAT RRADFNRGGG NGRGGRGRGG |
| 481 | PMGRGGYGGG GSGGGGRGGF PSGGGGGGGQ QRAGDWKCPN PTCENMNFSW RNECNQCKAP |
| 541 | KPDGPGGGPG GSHMGGNYGD DRRGGRGGYD RGGYRGRGGD RGGFRGGRGG GDRGGFGPGK |
| 601 | MDSRGEHRQD RRERLYEFGS GEGRGSLLTC GDVEENPGPM VSKGEEDNMA IIKEFMRFKV |
| 661 | HMEGSVNGHE FEIEGEGEGR PYEGTQTAKL KVTKGGPLPF AWDILSPQFM YGSKAYVKHP |
| 721 | ADIPDYLKLS FPEGFKWERV MNFEDGGVVT VTQDSSLQDG EFIYKVKLRG TNFPSDGPVM |
| 781 | QKKTMGWEAS SERMYPEDGA LKGEIKQRLK LKDGGHYDAE VKTTYKAKKP VQLPGAYNVN |
| 841 | IKLDITSHNE DYTIVEQYER AEGRHSTGGM DELYKZ |

1 . . . 85 = “TRIM21 RING 1-85”
91 . . . 616 = “FUS P525L”
619 . . . 639 = “T2A”
640 . . . 875 = “mCherry”

1. A fusion protein comprising:
(i) a first portion comprising a sequence of a protein which aggregates pathologically in neurodegenerative disease, and
(ii) a second portion comprising a RING-type E3 ubiquitin ligase, a component of a RING-type E3 ubiquitin ligase complex, or a domain of either.

2. A fusion protein as claimed in claim 1, wherein the second portion comprises said RING-type E3 ubiquitin ligase, or comprises said component or domain which possesses E3 ligase catalytic activity.

3. A fusion protein as claimed in any one of claims 1 to 2, wherein the component or domain comprises or is a RING domain.

4. A fusion protein as claimed in claim 3, wherein the component or domain comprises or is a RING-box.

5. A fusion protein as claimed in any one of claims 3 to 4, wherein the component or domain comprises or is derived from a polypeptide or RING domain shown in Table 1 and/or is derived from a TRIM polypeptide.

6. A fusion protein as claimed in any one of claims 2 to 5, wherein the RING domain comprises at least 30 amino acids.

7. A fusion protein as claimed in any one of claims 2 to 6, wherein the RING domain comprises SEQ ID No. 2 or a variant sharing at least 70% identity therewith.

8. A fusion protein as claimed in claim 1, wherein said component or domain is selected from: a scaffold component, an adaptor component, a substrate recognition component.

9. A fusion protein as claimed in claim 8, wherein the component is a VHL polypeptide, which optionally comprises SEQ ID No. 5 or a variant sharing at least 70% identity therewith.

10. A fusion protein as claimed in any one of claims 1 to 9, wherein the protein aggregation portion comprises a sequence of a protein selected from the list consisting of: tau; TDP43; alpha-synuclein; huntingtin; rhodopsin; super oxide dismutase; FUS protein.

11. A fusion protein as claimed in claim 10, wherein the protein aggregation portion comprises all or part of sequence shown in or described in Table 3 or of a variant sharing at least 70% identity therewith.

12. A fusion protein as claimed in any one of claims 1 to 11, wherein the protein aggregation portion comprises at least 25 amino acids.

13. A fusion protein as claimed in any one of claims 1 to 12, wherein the first portion is upstream of the second portion, from N terminus to C terminus.

14. A fusion protein as claimed in any one of claims 1 to 12, wherein the first portion is downstream of the second portion, from N terminus to C terminus.

15. A fusion protein as claimed in any one of claims 1 to 14, comprising one or more further amino acids or amino acid sequences between the first portion and the second portion, or at the N terminus or C terminus of the fusion protein.

16. A fusion protein as claimed in claim 15, comprising a linker between the first portion and the second portion.

17. A fusion protein as claimed in claim 16, wherein the linker comprises or consists of glycine and serine.

18. A fusion protein as claimed in any one of claims 1 to 12 which comprises SEQ ID No. 3 or 4 or a variant of either sharing at least 70% identity therewith.

19. A polynucleotide encoding the fusion protein of any one of claims 1 to 18.

20. A process for producing the polynucleotide of claim 19, the process comprising:
(a) providing a first nucleic acid encoding the first portion;
(b) providing a second nucleic acid encoding the second portion;
(c) combining said first and second nucleic acids.

21. A polynucleotide obtained or obtainable by the process of claim 20.

22. An expression vector comprising the polynucleotide of claim 19 or claim 21.

23. A vector as claimed in claim 22 wherein the polynucleotide encoding the fusion protein is operably linked to a promoter, which is optionally a tissue or cell specific promoter.

24. A vector as claimed in claim 23 wherein the promoter is a neuronal cell type-specific promoter.

25. A vector as claimed in any one of claims 22 to 24 wherein the vector is a viral vector.

26. A vector as claimed in claim 25 wherein the vector is an adenovirus vector and/or an adeno-associated vector (AAV), which is optionally selected from the group consisting of: AAV1, AAV2, AAV3, AAV4, AAV5, AAV6, AAV7, AAV8, AAV9, AAV10, AAV11, AAV 12, and hybrids or derivatives thereof, optionally selected from AAV-F, AAV-9P31 and AAV-9P801.

27. A vector as claimed in claim 25 wherein the vector is a herpes virus vector, a retrovirus vector, or a lentivirus vector.

28. A host cell comprising, or transfected with, or transformed with, the polynucleotide or vector of claim 19 or 21, or any one of claims 22 to 27.

29. A process for producing the fusion protein of any one of claims 1 to 18, the method comprising:
(a) stably transforming a host cell with the expression vector of any one of claims 22 to 27;
(b) cultivating said transformed host cell under conditions suitable for said host cell to produce said fusion protein; and
(c) optionally recovering said fusion protein.

30. A fusion protein obtained or obtainable by the process of claim 29.

31. A viral particle comprising a vector of any one of claims 25 to 27.

32. A pharmaceutical composition comprising an agent which is a fusion protein, polynucleotide, vector or particle of any one of claims 1 to 19 or 21 to 27 or 30 to 31, plus optionally a pharmaceutically acceptable carrier, diluent, or excipient.

33. A method of inhibiting formation of, or increasing degradation or clearance of, pathological aggregates of an aggregating protein in a cell,
which method comprises introducing a fusion protein of any one of claims 1 to 18, or claim 30 into the cell,
optionally via expression from a polynucleotide, vector or particle of any one of claim 19, 21 to 27, or 31,
such that a plurality of said fusion protein molecules are incorporated into a co-aggregate with said aggregating protein,
and said plurality of fusion proteins cluster in the co-aggregate, causing ubiquitination of the co-aggregate,
thereby leading to inhibited formation of aggregates, or increased degradation or clearance of aggregates, in the cell.

34. A method as claimed in claim 33 wherein the cell is present in an organism, which is optionally a non-human test animal.

35. A method of treating a neurodegenerative disease in a subject, wherein the neurodegenerative disease is associated with a protein which aggregates pathologically, the method comprising:
(1) providing an agent selected which is a fusion protein, polynucleotide, vector, particle, or composition of any one of claim 1 to 19 or 21 to 27 or 30 to 31 or 32;
(2) administering said agent to said subject

36. The method of claim 35 which comprises the steps of:
(i) assembling viral particles of claim 28 in vitro by transducing mammalian cells with the expression vector and expressing viral packaging and envelope proteins necessary for particle formation in the cells and culturing the transduced cells in a culture medium, such that the cells produce viral particles comprising the expression vector that are released into the medium;
(ii) administering the viral particles to the subject.

37. A method as claimed in claim 35 or claim 36 wherein the subject is a human subject.

38. A method as claimed in any one of claims 33 to 37, wherein the protein of the first portion of the fusion protein and the respective disease are as defined in Table 2, optionally wherein the protein which aggregates pathologically is tau protein, and the disease is Alzheimer's disease or Progressive supranuclear palsy, or wherein the protein which aggregates pathologically is Fused in sarcoma (FUS) protein and the disease is amyotrophic lateral sclerosis or frontotemporal dementia.

39. A method as claimed in any one of claims 35 to 37, wherein the subject is a non-human mammal, bird, fish, reptile or amphibian.

40. An agent as defined in claim 35 for use in a method of treatment of the human or animal body.

41. An agent for use as claimed in claim 40, wherein the treatment is of neurodegenerative disease in a subject.

42. Use of an agent as defined in claim 35 in the manufacture of a medicament for the treatment of neurodegenerative disease in a subject.

43. An agent for use, or use, as claimed in any one of claims 40 to 42, wherein the treatment is as defined in any one of claims 35 to 39.


SEQUENCE LISTING (33 sequences)

SEQ ID NO: 1 - protein, 475 aa, Homo sapiens [sequence omitted]

SEQ ID NO: 2 - protein, 85 aa, Homo sapiens
MASAARLTMMWEEVTCPICLDPFVEPVSIECGHSFCQECISQVGKGGGSVCPVCRQRFLLKNLRPNRQLANMVNNLKEISQEARE

SEQ ID NO: 3 - protein, 477 aa, synthetic construct
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVSGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLEFGSGGGSGMASAARLTMMWEEVTCPICLDPFVEPVSIECGHSFCQECISQVGKGGGSVCPVCRQRFLLKNLRPNRQLANMVNNLKEISQEARE

SEQ ID NO: 4 - protein, 718 aa, synthetic construct
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVSGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLEFGSMVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAAGITLGMDELYKGSGGGSGMASAARLTMMWEEVTCPICLDPFVEPVSIECGHSFCQECISQVGKGGGSVCPVCRQRFLLKNLRPNRQLANMVNNLKEISQEARE

SEQ ID NO: 5 - protein, 213 aa, synthetic construct
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 6 - protein, 43 aa, synthetic construct
QKVDHCARHGEKLLLFCQEDGKVICWLCERSQEHRGHHTFLTE

SEQ ID NO: 7 - protein, 876 aa, synthetic construct
MASAARLTMMWEEVTCPICLDPFVEPVSIECGHSFCQECISQVGKGGGSVCPVCRQRFLLKNLRPNRQLANMVNNLKEISQEAREGGGGSMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERLYEFGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKZ

SEQ ID NO: 8 - protein, 57 aa, Homo sapiens
SGTVSCPICMDGYSEIVQNGRLIVSTECGHVFCSQCLRDSLKNANTCPTCRKKINHK

SEQ ID NO: 9 - protein, 64 aa, Homo sapiens
EISTEEQLRRLQEEKLCKICMDRNIAIVFVPCGHLVTCKQCAEAVDKCPMCYTVITFKQKIFMS

SEQ ID NO: 10 - protein, 49 aa, Homo sapiens
EVTCPICLELLTQPLSLDCGHSFCQACLTANHKKSMLDKGESSCPVCRI

SEQ ID NO: 11 - protein, 71 aa, Homo sapiens
QGEATCSICLELFREPVSVECGHSFCRACIGRCWERPGAGSVGAATRAPPFPLPCPQCREPARPSQLRPNR

SEQ ID NO: 12 - protein, 57 aa, Homo sapiens
IEPCVICQGRPKNGCIVHGKTGHLMACFTCAKKLKKRNKPCPVCRQPIQMIVLTYFP

SEQ ID NO: 13 - protein, 77 aa, Homo sapiens
QDHIKVTQEQYELYCEMGSTFQLCKICAENDKDVKIEPCGHLMCTSCLTSWQESEGQGCPFCRCEIKGTEPIVVDPF

SEQ ID NO: 14 - protein, 93 aa, Homo sapiens
RVEEVQNVINAMQKILECPICLELIKEPVSTKCDHIFCKFCMLKLLNQKKGPSQCPLCKNDITKRSLQESTRFSQLVEELLKIICAFQLDTGL

SEQ ID NO: 15 - protein, 71 aa, Homo sapiens
IPDYLCGKISFELMREPCITPSGITYDRKDIEEHLQRVGHFDPVTRSPLTQEQLIPNLAMKEVIDAFISEN

SEQ ID NO: 16 - protein, 62 aa, Homo sapiens
DNCAICRNHIMDLCIECQANQASATSEECTVAWGVCNHAFHFHCISRWLKTRQVCPLDNREW

SEQ ID NO: 17 - protein, 75 aa, Homo sapiens
LTMMWEEVTCPICLDPFVEPVSIECGHSFCQECISQVGKGGGSVCPVCRQRFLLKNLRPNRQLANMVNNLKEISQ

SEQ ID NO: 18 - protein, 86 aa, Homo sapiens
AWAHSRAALDRLEKLLRCSRCTNILREPVCLGGCEHIFCSNCVSDCIGTGCPVCYTPAWIQDLKINRQLDSMIQLCSKLRNLLHDN

SEQ ID NO: 19 - protein, 55 aa, Homo sapiens
GSDEECAICLDSLTVPVITHCAHVFCKPCICQVIQNEQPHAKCPLCRNDIHEDNL

SEQ ID NO: 20 - protein, 54 aa, Homo sapiens
YECMVCCELVRVTAPVWSCQSCYHVFHLNCIKKWARSPASQADGQSGWRCPACQ

SEQ ID NO: 21 - protein, 56 aa, Homo sapiens
FLACKICLEQLRAPKTLPCLHTYCQDCLAQLADGGRVRCPECRETVPVPPEGVASF

SEQ ID NO: 22 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 23 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 24 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 25 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 26 - protein, 348 aa, Homo sapiens [sequence omitted]

SEQ ID NO: 27 - protein, 154 aa, Homo sapiens [sequence omitted]

SEQ ID NO: 28 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 29 - protein, 7 aa, synthetic construct
GSGGGSG

SEQ ID NO: 30 - protein, 4 aa, synthetic construct
GSSS

SEQ ID NO: 31 - protein, 12 aa, synthetic construct
GSSSGSSSGSSS

SEQ ID NO: 32 - protein, 109 aa, synthetic construct
  VARIANT 2..3: note = X can be any amino acid
  VARIANT 5..43: note = Upto thirty X residues may be absent
  VARIANT 44: note = X can be C or H
  VARIANT 45..48: note = Upto three X residues may be absent
  VARIANT 49: note = X can be H,N or C
  VARIANT 50..53: note = Upto two X residues may be absent
  VARIANT 54: note = X can be C or H
  VARIANT 55..56: note = X can be any amino acid
  VARIANT 58..105: note = Upto fourty four X residues may be absent
  VARIANT 107..108: note = X can be any amino acid
  VARIANT 109: note = X can be C or D
  VARIANT 5..43: note = X can be any amino acid
  VARIANT 58..105: note = X can be any amino acid
  VARIANT 45..48: note = X can be any amino acid
  VARIANT 50..53: note = X can be any amino acid
CXXCXXXXXXXXXXXXXXXXXXXXXXXXXXXXXXXXXXXXXXXXXXXXXXXXXXXXCXXXXXXXXXXXXXXXXXXXXXXXXXXXXXXXXXXXXXXXXXXXXXXXXCXXX

SEQ ID NO: 33 - protein, 107 aa, synthetic construct
  VARIANT 2..3: note = X can be any amino acid
  VARIANT 5..43: note = Upto Thirty X residues may be absent
  VARIANT 45..47: note = Upto two X residues may be absent
  VARIANT 49..51: note = Upto one X residue may be absent
  VARIANT 53..54: note = X can be any amino acid
  VARIANT 56..103: note = Upto fourty four X residues may be absent
  VARIANT 105..106: note = X can be any amino acid
  VARIANT 5..43: note = X can be any amino acid
  VARIANT 45..47: note = X can be any amino acid
  VARIANT 56..103: note = X can be any amino acid
  VARIANT 49..51: note = X can be any amino acid
CXXCXXXXXXXXXXXXXXXXXXXXXXXXXXXXXXXXXXXXXXXCXXXHXXXCXXCXXXXXXXXXXXXXXXXXXXXXXXXXXXXXXXXXXXXXXXXXXXXXXXXCXXC